(19) United States
(12) Patent Application Publication
(10) Pub. No.: US 2026/0265690 A1
(43) Pub. Date: Sep. 10, 2026

(54) Epithelial Stem Cells and Production and Uses thereof

(71) Applicant: Stemson Therapeutics Corp., San Diego, CA (US)

(72) Inventors: Yulia Shamis, San Diego, CA (US); Antonella Pinto, San Diego, CA (US); Alexey Terskikh, San Diego, CA (US)

(73) Assignee: Stemson Therapeutics Corp., San Diego, CA (US)

(21) Appl. No.: 18/869,354

(22) PCT Filed: Jun. 2, 2023
(86) PCT No.: PCT/US23/24271

§ 371 Date: Nov. 26, 2024

Related U.S. Application Data
(60) Provisional application No. 63/348,882, filed on Jun. 3, 2022.

(51) Int. Cl. C12N 5/071; A61K 35/36; A61P 17/02; C07K 14/47

(52) U.S. Cl. CPC C12N 5/0625; A61K 35/36; A61P 17/02; C07K 14/4741; C07K 14/4748; C12N 2500/90; C12N 2501/11; C12N 2501/115; C12N 2501/15; C12N 2501/155; C12N 2501/385; C12N 2501/727; C12N 2506/45; C12N 2533/52; C12N 2533/54

(57) ABSTRACT

Provided herein is method for producing epithelial stem cells (EPSCs) from human induced pluripotent stem cells. Provided herein are also methods for culturing the EPSCs, and methods of producing or growing organoids, hair follicles, interfollicular epidermis, or skin grafts using the EPSCs.

### CROSS REFERENCE TO RELATED APPLICATIONS

This application claims the benefit of priority of U.S. Provisional Application No. 63/348,882, filed Jun. 3, 2022, which is incorporated by reference herein in its entirety for any purpose.

### FIELD OF THE INVENTION

The present disclosure provides methods related to producing epithelial stem cells (EPSCs) from human induced pluripotent stem cells (hiPSCs), EPSCs produced by the claimed methods, methods of culturing the EPSCs, organoids produced using the EPSCs, and methods of producing or growing hair follicles, interfollicular epidermis, or skin grafts using the EPSCs.

### INTRODUCTION AND SUMMARY

Hair follicles (HF) are characterized by the ability to shed and produce new hair shafts through repetitive cycling. This cycling consists of three different phases, where the duration of each phase differs based on the location of the HF and communication between specific cell types within the HF. However, for a large proportion of the global population, some HFs undergo premature degeneration or miniaturization leading to permanent hair loss. Hair loss is common and occurs in a variety of physiological states associated with hormonal imbalance, age, autoimmune conditions, medications, and genetics. Chen et al., J Dermatol Sci. 66(1):3-11 (2012). Damaged or destroyed HF stem cells are thought to be the reason underlying hair loss across these different situations. Matsumura et al., Science 351: aad4395 (2016). The sociocultural importance of hair in human society is substantial; therefore, those hair loss disorders are often interrelated with mental health. This also holds true for patients with severe burns, scarring, and injuries who have lost their HFs. Beyond the aesthetic value, it has been shown that HFs play an important role in physical, chemical, and biological functions of normal skin. Previous studies have shown that hair-bearing skin heals more quickly and exhibits minimal scarring. See, e.g., Ito et al., Nat. Med. 11:1351-1354 (2005); Levy et al., FASEB J. 21:1358-1366 (2007); Ge et al., Cell 169: 636-650.e14 (2017)). These observations suggested that optimal wound healing processes depend on intact HFs.

Currently, there is no definitive treatment for hair loss and few available treatments to halt or reverse miniaturization. While some progress has been made towards treating alopecia with autologous hair transplantation, light therapy, or drugs, most efforts currently only help manage hair loss. Both locally applied (minoxidil/ROGAINE®) and systemic (finasteride/PROPECIA®) drugs achieve only limited success in hair regeneration and mainly assist in slowing additional hair loss. Price, V., N. Engl. J. Med., 1999. 341:964-973. Other hair restoration procedures involve the excision of existing hair follicles and re-implantation of these follicles into thinning areas. However, the number of follicles that can be harvested and re-implanted is limited, and restoration is likely temporary due to the progressive nature of hair loss conditions. Zhang et al., J Tissue Eng, 2014. 5:2041731414556850. Given the limited efficacy of strategies currently available in hair loss management, better solutions are urgently needed.

During formation of a new HF, dermal papilla (DP) cells located at the base of the HF interact with epithelial cells responsible for forming the multiple concentric layers that comprise the hair shaft. During the natural HF life cycle, these epithelial layers undergo regression and regrowth stages, fueled by the HF epithelial stem cells (EPSCs), located in the bulge region in the outer root sheath (ORS). See Rishikaysh et al., Int J Mol Sci. 2014, 15(1):1647-1670; Houschyar et al., Dermatology. 2020, 236(4):271-280. HF bulge EPSCs express markers such as KRT15 (keratin 15), KRT19 (keratin 19), and genes associated with Wnt, BMP (bone morphogenetic protein), and TGF-β (transforming growth factor-beta) signaling pathways, including Lef1 (lymphoid enhancer-binding factor 1), TCF4 (transcription factor 4), DKK3 (Dickkopf-related protein 3), FZD1 (frizzled-1), LGR6 (leucine-rich repeat containing G-protein coupled receptor 6), FST (follistatin), and TGFβ2 (transforming growth factor-beta 2). Ohyama et al., J Clin Invest. 2006 January, 116(1):249-60; Merril et al., Genes Dev. 2001 Jul. 1, 15(13):1688-705; Rishikaysh et al., Int J Mol Sci. 2014, 15(1):1647-1670. Human HF bulge EPSCs have been shown to express cell-surface markers CD200, CD59, and CD99. Ohyama et al., J Clin Invest. 2006 January, 116(1):249-60; Inoue et al., Lab Invest. 2009 August, 89(8):844-56; Choi et al., J Pathol Transl Med. 2016 September, 50(5): 361-8. However, when isolated using cell-surface markers, HF bulge EPSCs cannot maintain the self-renewal ability in vitro and rapidly lose their stem cell marker expression and function. See, e.g., Ohyama et al., J Clin Invest. 2006 January, 116(1):249-60; Inoue et al., Lab Invest. 2009 August, 89(8):844-56; Yang et al., Stem Cell Research & Therapy (2020) 11:303.

Described herein is an alternative method for generating folliculogenic EPSCs from human induced pluripotent stem cells (hiPSC) that can reconstitute both the epithelial components of HFs and interfollicular epidermis. Using the disclosed methods, CD59 positive EPSCs were obtained from hiPSC (hiPSC-EPSCs) that can be expanded for multiple passages in vitro while maintaining their stem cell marker expression and functionality. These hiPSC-EPSCs can have high colony forming efficiency and show a gene expression signature similar to human bulge EPSC. It is further demonstrated that iPSC-EPSCs can be receptive to specific mesenchymal stimuli and capable of creating complex multilayered tissues in functional bioassays, both in vitro and in vivo. When co-cultured with dermal papilla cells in a 3D HF organoid assay, iPSC-EPSCs recapitulated the key events of the HF morphogenesis by inducing an architecture similar to that of a natural HF. While not required for positive outcomes, the use in culture of a specific GSK-3 inhibitor and Wnt agonist (CHIR-99021) and not BMP or TGF-β inhibitors (DMH-1 and A-83-01, respectively) with the hiPSC-EPSCs was shown to improve the colony forming efficiency and HF organoid assay outcomes. When injected into the skin of immunodeficient mice in combination with mouse neonatal dermal cells, iPSC-EPSC regenerated the hair follicles and contributed to the interfollicular epidermis of the hair patch. When co-cultured with dermal fibroblasts in a 3D skin equivalent assay, iPSC-EPSCs formed multilayered epithelial tissue and expressed epidermal differentiation markers.

The results indicate that hiPSC-derived CD59-positive and CD99-positive epithelial cells are molecularly and functionally similar to human HF bulge EPSCs. These results suggest that the methods for generating hiPSC-EPSCs described herein can be used to generate a substantial number of folliculogenic EPSCs for use in therapeutic applications.

The following exemplary embodiments are provided.

In some embodiments, the disclosure provides a method of producing epithelial stem cells (EPSCs) from human induced pluripotent stem cells (hiPSCs), wherein the method comprises:
(a) culturing hiPSCs in a medium comprising retinoic acid (RA);
(b) culturing the cells of step (a) in a medium comprising RA, bone morphogenetic protein 4 (BMP4), and epidermal growth factor (EGF);
(c) culturing the cells of step (b) in a medium comprising a keratinocyte serum-free medium, RA, BMP4, EGF, and ROCK inhibitor; and
(d) culturing the cells of step (c) in a medium comprising a keratinocyte serum-free medium, BMP4, EGF, and Rock inhibitor, thereby producing EPSCs.

In some embodiments, the culturing steps (a), (b), (c), and (d) are conducted for a duration independently selected from about 1, 2, 3, or more days.

In some embodiments, the culturing steps (a), (b), (c), and (d) are each conducted for 2 days.

In some embodiments, the disclosure provides a method of producing epithelial stem cells (EPSCs) from human induced pluripotent stem cells (hiPSCs), wherein the method comprises:
(a) culturing hiPSCs in a medium comprising retinoic acid (RA) for 2 days;
(b) culturing the cells of step (a) in a medium comprising RA, bone morphogenetic protein 4 (BMP4), and epidermal growth factor (EGF) for 2 days;
(c) culturing the cells of step (b) in a medium comprising RA, BMP4, EGF, and ROCK inhibitor for 2 days; and
(d) culturing the cells of step (c) in a medium comprising BMP4, EGF, and Rock inhibitor for 2 days.

In some embodiments, the medium of culturing step (a) does not include BMP4.

In some embodiments, the medium of culturing step (a) further comprises a keratinocyte serum-free medium and a matrix comprising a solubilized basement membrane matrix secreted by Engelbreth-Holm-Swarm (EHS) mouse sarcoma cells and human Collagen Type I (BMM/Coll1 matrix).

In some embodiments, the medium of culturing step (b) further comprises the BMM/Coll1 matrix.

In some embodiments, the medium of culturing step (c) further comprises human Collagen type I, fibronectin, and laminin-521 (Coll1/FN/LN521).

In some embodiments, the medium of culturing step (d) further comprises Coll1/FN/LN521.

In some embodiments, the medium used in culturing steps (a)-(d) is feeder-free and serum-free.

In some embodiments, the disclosure provides a method of producing epithelial stem cells (EPSCs) from human induced pluripotent stem cells (hiPSCs) comprising:
differentiating hiPSCs into EPSCs using a feeder-free and serum-free system comprising retinoic acid (RA), bone morphogenetic protein 4 (BMP4), epidermal growth factor (EGF), and ROCK inhibitor.

In some embodiments, the EPSCs express TP63 (tumor protein p63) and KRT15 (keratin 15).

In some embodiments, the method further comprises culturing the hiPSCs for one or more days with:
i. a BMM/Coll1 matrix; and
ii. a serum-free medium comprising basal medium, recombinant human basic fibroblast growth factor (rh bFGF), and recombinant human transforming growth factor β (rh TGFβ).

In some embodiments, the method further comprises expanding the EPSCs by maintaining the cells on a feeder-free medium.

In some embodiments, the feeder-free maintaining step comprises:
(a) culturing in a first feeder-free medium comprising BMP4, EGF, and ROCK inhibitor; and
(b) maintaining the cells of step (a) in a second feeder-free medium comprising EGF and ROCK inhibitor, optionally wherein the second feeder-free medium does not comprise BMP4.

In some embodiments, culturing step (a) and maintaining step (b) are each conducted for 1, 2, 3, or more days, which may be the same or different number of days.

In some embodiments, culturing step (a) and maintaining step (b) are both conducted for 2 days.

In some embodiments, the feeder-free medium of culturing step (a) further comprises Coll1/FN/LN521 and a keratinocyte serum-free medium.

In some embodiments, the feeder-free medium of maintaining step (b) further comprises a keratinocyte serum-free medium.

In some embodiments, the expanding step is for at least 2, least 3, at least 4, at least 5, or at least 6 passages.

In some embodiments, the expanding step is for 6 passages.

In some embodiments, the method further comprises expanding the EPSCs by maintaining the cells on a feeder medium comprising mitotically inactivated 3T3-J2 mouse fibroblasts.

In some embodiments, the feeder medium further comprises ROCK inhibitor.

In some embodiments, the feeder medium further comprises Coll1/FN/LN521 and a keratinocyte growth medium.

In some embodiments, the keratinocyte growth medium comprises DMEM/F12, dipeptide L-alanine-L-glutamine, keratinocyte growth medium 2 (KG2) basal medium, fetal bovine serum (FBS), adenine, an effective amount of one or more antibiotics and/or antimycotics, hydrocortisone, cholera toxin (CT), epidermal growth factor (EGF), and insulin.

In some embodiments, the feeder medium further comprises DMEM, bovine calf serum (BCS), one or more antibiotics and/or antimycotics, and L-Glutamine.

In some embodiments, the one or more antibiotics and/or antimycotics comprise penicillin, streptomycin, and amphotericin B.

In some embodiments, the EPSCs have increased expression of markers TP63, KRT14, and KRT15 after/during expansion as compared to iPSC controls.

In some embodiments, the BMM/Coll1 matrix is formed by:
combining BMM with DMEM/F12 to form a diluted BMM solution;
adding human Collagen Type I (Coll1) solution to the diluted BMM solution to form a BMM/Coll1 matrix.

In some embodiments, the method further comprises culturing the EPSCs with a perturbagen.

In some embodiments, the perturbagen is a GSK-3 inhibitor and/or Wnt agonist.

In some embodiments, the GSK-3 inhibitor and/or Wnt agonist is CHIR-99021.

In some embodiments, the EPSC expresses at least two, three, four, or each of CD59, CD99, LEF1, CTNNB1, FZD1, LGR5, DCT, TCF4, and LGR6.

In some embodiments, the EPSC expresses at least two, three, four, five, six, seven, eight, nine, or each of CD59, CD99, KRT15, KRT19, DKK3, FZD1, FZD2, CTNNB1, TGFB2, FST, DCN, SOX9, NFATC1, ITGA6, ITGB1, LEF1, TCF4, TP63, and LGR6.

In some embodiments, the disclosure provides an epithelial stem cell (EPSC) produced by any of the methods described herein.

In some embodiments, the EPSC expresses one or more of CD59, CD200, KRT15, KRT19, DKK3, FZD1, FZD2, CTNNB1, TCF4, LEF1, LGR6, TGFB2, SOX9, NFATC1, and DCN at an increased level relative to a naturally occurring EPSC, and/or one or more of ITGA6, ITGB1, FST, KRT14 and TP63 at a decreased level relative to a naturally occurring EPSC.

In some embodiments, the disclosure provides an EPSC differentiated from an induced pluripotent stem cell, wherein the EPSC expresses one or more of CD59, CD200, KRT15, KRT19, DKK3, FZD1, FZD2, CTNNB1, TCF4, LEF1, LGR6, TGFB2, and DCN at an increased level relative to a naturally occurring EPSC, and/or one or more of ITGA6, ITGB1, FST, KRT14, and TP63 at a decreased level relative to a naturally occurring EPSC.

In some embodiments, the EPSC expresses two, three, four, five, six, seven, eight, nine, ten, or each of CD59, CD200, KRT15, KRT19, DKK3, FZD1, FZD2, CTNNB1, TCF4, LEF1, LGR6, TGFB2, and DCN at an increased level relative to a naturally occurring EPSC.

In some embodiments, the EPSC expresses two, three, or each of ITGA6, ITGB1, FST, KRT14, and TP63 at a decreased level relative to a naturally occurring EPSC.

In some embodiments, the naturally occurring EPSC is a bulge hair follicle-derived keratinocyte.

In some embodiments, the EPSC is a human EPSC.

In some embodiments, the disclosure provides a method of culturing the EPSC under conditions to obtain hair follicles and/or interfollicular epidermis.

In some embodiments, the disclosure provides an organoid produced using the EPSCs.

In some embodiments, the organoid is a folliculogenic organoid.

In some embodiments, the disclosure provides a method of producing hair follicles comprising culturing the EPSCs with fibroblasts and/or dermal papilla cells to obtain hair follicles.

In some embodiments, the disclosure provides a method of producing a skin graft comprising culturing the EPSCs under conditions to produce a skin graft.

In some embodiments, the disclosure provides a method of producing a skin graft comprising culturing the EPSCs under conditions to produce a cultured epidermal graft.

In some embodiments, the disclosure provides for use of the EPSCs to obtain hair follicles and/or interfollicular epidermis.

In some embodiments, the disclosure provides for use of the EPSCs for a replacement skin graft.

In some embodiments, the method or use comprises delivering the EPSCs onto a wound bed, optionally wherein delivering comprises spraying.

In some embodiments, the EPSCs are expanded in culture, optionally wherein the expansion occurs before delivering the EPSCs onto a wound bed.

In some embodiments, the EPSCs are cultured with a dermal component, optionally wherein the dermal component comprises dermal fibroblasts and/or a collagen substrate.

In some embodiments, the disclosure provides a method of growing a hair follicle, comprising:
loading one or more EPSCs into a cellular scaffold; and
implanting the cellular scaffold into dermal tissue.

In some embodiments, the cellular scaffold is in the form of a hydrogel, a fiber network, or a photoinitiated 3D structure.

In some embodiments, the cellular scaffold comprises a biodegradable polymer.

In some embodiments, the cellular scaffold comprises:
a cell reservoir; and
a guide attached to the cell reservoir comprising one or more first biodegradable polymers.

In some embodiments, the disclosure provides a method of growing a hair follicle, comprising implanting the organoid into dermal tissue.

In some embodiments, the method further comprises piercing the dermal tissue with a needle prior to implanting the organoid.

In some embodiments, a cellular scaffold containing the organoid is implanted into the dermal tissue.

### BRIEF DESCRIPTION OF THE DRAWINGS

FIGS. 1A-1C show generation of EPSCs from hiPSC. FIG. 1A shows schematic representation of the differentiation protocol and the factors administered at each stage. EPSCs emerge on day 8 of differentiation and can be further expanded in the presence of Y-27632 either in the serum-free/feeder-free conditions or on a feeder layer of mitotically inactivated 3T3-J2 mouse fibroblasts. FIG. 1B shows transcript expression, measured by bulk RNASeq, of pluripotency markers (NANOG, POU5F1 (OCT3/4), TERT, and CD200), epithelial lineage markers (KRT19 and KRT15), the non-neural ectoderm marker TFAP2A, the neural ectoderm marker FOXG1, and EPSC markers (CD99, LEF1, CTNNB1 (β-Cat), FZD1, LGR5, and DCT) at different stages of differentiation. Samples were collected at day 0 (iPSC), day 4 (EP-D4) and day 8 (EP-D8) of differentiation. Data are represented as a heatmap, where results from eight independent differentiation experiments were averaged and z-scores were calculated for each timepoint represented. FIG. 1C. Cell morphology changes during the differentiation of EPSC from hiPSC (D0-D8) and the expansion process in feeder-free or feeder-based culture.

FIGS. 2A-2D show characterization of hiPSC-EPSC in vitro. FIG. 2A shows cumulative population doublings (CPD) calculated based on the cell counts obtained during culture expansion of three different EPSC lines (EP61, EP112 and EP116) compared to control hair follicle derived keratinocytes (HF-KC). FIG. 2B shows representative images of colony formation assays (CFA) of hiPSC-EPSCs expanded in culture for three weeks. For CFA, different hiPSC-EPSC lines (EP61, EP112 and EP116) and control HF-KC were subcultured for 3 passages (p3) then plated on 3T3 fibroblast feeders at 1K/well and incubated for additional 10 days, then fixed and stained with Crystal violet. FIG. 2C shows the differentiation trajectory, analyzed by principal component analysis, of three different iPSC lines to EPSC (EP61 (61), EP112 (112), and EP116 (116)). Hair follicle derived keratinocytes (HFKC) are used as reference cells. FIG. 2D shows transcript-level expression, measured by bulk RNASeq, of HF bulge markers including CD59, CD200, ITGA6, ITGB1, KRT15, KRT19, KRT15, TCF4, LEF1, CTNNB1, DKK3, FZD1, FZD2, LGR6, TGFB2, FST, DCN, TP63, and KRT14 in iPSC, passage 4 EPSC (EP-p4), and hair follicle derived keratinocytes (HFKC). Data represent average values from eight independent differentiation experiments and values are displayed as the log 2 of the transcripts per million (TPM) for each gene. Ns: p>0.05; *: p<0.05; ** p<0.01; *** p<0.001; ****: p<0.0001. FIG. 2E shows immunofluorescence analysis of KRT15, KRT19, p63, KRT14, beta-Cat (i.e., CTNNB1 or β-Cat), LEF1, and Ki67 in hiPSC-EPSC and HF-KC culture. Note nuclear localization of 3-Cat, a hallmark of Wnt signaling activation in hiPSC-EPSC but not in HF-KC (arrows).

FIGS. 3A-3E show activation of Wnt signaling pathway via GSK3 inhibition enables long-term expansion of hiPSC-EPSC. FIG. 3A shows representative images of colony formation assays of hiPSC-EPSCs expanded in culture for 2 to 5 weeks (p2-p5). The clonogenicity of hiPSC-EPSCs was diminishing as a result of serial passaging. FIG. 3B shows representative images of colony formation assays of hiPSC-EPSCs treated with different combinations of CHIR-99021, DMH-1, and A-83-01 inhibitors (left panel). For CFA, hiPSC-EPSCs were cultured for 7 weeks (p7) with or without small molecule inhibitors, then plated on 3T3 fibroblast feeders at 1K/well and incubated for additional 10 days, then fixed and stained with Crystal violet. The plates were scanned and analyzed using imaging software to determine the confluency. The confluency data from three experiments is summarized in the graph (right panel). FIG. 3C shows representative images of immunofluorescent analysis of proliferation marker, Ki67 (white) in hiPSC-EPSC treated with CHIR-99021 in comparison to untreated cells. The graph is showing the percentage of proliferating (Ki67+) hiPSC-EPSC over total nuclei (DAPI+). *: p<0.05. FIG. 3D shows gene expression analysis by qPCR of Wnt signaling pathway associated genes LEF1, TCF4 and LGR6. Data was analyzed using standard ddCt method and shown as mean±s.d. of the expression from three independent experiments. *: p<0.05; ****: p<0.01. FIG. 3E shows transcript expression of EPSC markers (KRT15, CD59, TP63, ITGA6, DKK3, SOX9, and NFATC1) and CHIR targets (MYC, and CCND1) in passage 1 (EP-p1), passage 2 (EP-p2), passage 3 (EP-p3), and passage 4 EPSC treated with DMSO (EP-p4) or CHIR-99021 (EP-p4-CHIR). Data are represented as a heatmap, where results from eight independent differentiation experiments were averaged and z-scores were calculated for each timepoint represented.

FIGS. 4A-4D show in vitro functional analysis of hiPSC-EPSC in 3D tissue constructs. FIG. 4A shows a graphical schematic of in vivo HF morphogenesis (top panel) highlighting the similarities with the stages observed during the development of HF organoids in vitro. Bottom panel illustrates an organoid development process of using an example of an organoid created by combining fluorescently labeled (red) dermal papilla (DP) cells with HF-KCs. FIG. 4B shows representative images of the key steps of organoids development obtained combining hiPSC-EPSC with DP cells. FIG. 4C shows a measurement system to evaluate organoids elongation by plotting the number of organoids that meet the criteria of round, polarized or elongated. Measurement is obtained with CYTATION™ 5 software. Each color represents a different cell combination, and each dot represents a single organoid. The percentage of elongated organoids was increased when hiPSC-EPSC were treated with CHIR-99021. FIG. 4D shows differentiation of hiPSC-EPSC into mature keratinocytes in vitro in human skin equivalent assay. Neonatal foreskin-derived keratinocytes (HFK) were used as a control. The 3D skin equivalents exhibited a multilayered epidermis (H&E) and expressed differentiation markers of stratified squamous epithelia (KRT10 (listed as K10) and KRT14 (listed as K14)), and proliferation marker of basal keratinocytes (Ki67).

FIGS. 5A-5E show the folliculogenic potential of hiPSC-EPSC in vivo. FIG. 5A shows hiPSC-EPSC form HFs in mouse hair patch assay. hiPSC-EPSCs and HF-KC were combined with mouse neonatal dermal cells (mDC) and injected into the dermis of an immunodeficient mouse. FIG. 5B shows representative images of hair patches formed at the site of injection in the host subcutis 3 weeks after transplantation. FIG. 5C shows representative image of histological analysis of a hair patch formed by hiPSC-EPSC combined with mDC (mDC+EP) illustrating that injected epithelial cells aggregated to form a cyst in the host subcutis. Hair follicles growing outward from the cyst were evident. FIG. 5D shows representative stereoscopic images of the hair patches formed using different combinations of mDC and hiPSC-EPSC or HF-KC, mDC injected alone was used as negative control for hair patch development. FIG. 5D shows quantitative Alu-qPCR assay with a human-specific Alu-repeat sequence primer shows that patches formed with hiPSC-EPCS contain higher number of human cells when compared to patches formed with HF-KC. FIG. 5E shows immunofluorescent analysis of reconstituted hair follicles formed by hiPSC-EPSCs or HF-KC performed using antibodies against human specific KRT14 and human-specific nuclear protein Ku80.

### DETAILED DESCRIPTION OF CERTAIN EMBODIMENTS

Reference will now be made in detail to certain embodiments of the invention. While the invention will be described in conjunction with such embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a nucleic acid” includes a plurality of nucleic acids, reference to “a cell” includes a plurality of cells, and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes and are not to be construed as limiting the disclosed subject matter in any way. In the event that any document or other material incorporated by reference contradicts any explicit content of this specification, including definitions, this specification controls.

### I. Definitions

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All references cited herein are incorporated by reference in their entirety as though fully set forth. In case of any contradiction or conflict between material incorporated by reference and the expressly described content provided herein, the expressly described content controls. Singleton et al., Dictionary of Microbiology and Molecular Biology 3rd ed., J. Wiley & Sons (New York, NY 2001); March, Advanced Organic Chemistry Reactions, Mechanisms and Structure 5th ed., J. Wiley & Sons (New York, NY 2001); and Sambrook and Russel, Molecular Cloning: A Laboratory Manual 3rd ed., Cold Spring Harbor Laboratory Press (Cold Spring Harbor, NY 2001), provide one skilled in the art with a general guide to many of the terms used in the present application.

As used herein, an “epithelial stem cell (EPSC)” refers to a multipotent cell which has the potential to become committed to multiple cell lineages, including cell lineages resulting in epithelial cells. In some embodiments, the EPSCs have increased expression of markers including, but not limited to, TP63 (tumor protein p63), KRT14, and KRT15 (keratin 15), e.g., relative to induced pluripotent stem cells (iPSCs).

A “pluripotent stem cell” as used herein refers to a stem cell that can be cultured in vitro and has the ability to be differentiated into any of the three germ layers (ectoderm, mesoderm, endoderm). The pluripotent stem cell may be a non-genetically modified pluripotent stem cell or a genetically modified pluripotent stem cell. The pluripotent stem cells may be mammal-derived pluripotent stem cells, such as from rodents and primates, for example where the primates are humans.

As used herein, the term “induced pluripotent stem cells” or “iPSCs,” also commonly abbreviated as “iPS cells”, refers to pluripotent stem cells artificially derived from a non-pluripotent cell, such as an adult somatic cell, by artificially inducing expression of certain genes. In some embodiments, human induced pluripotent stem cells (hiPSCs) are utilized.

“Mammals” include, but are not limited to, rodents, ungulates, carnivora, primates, and the like. Rodents include mice, rats, hamsters, guinea pigs, and the like. Ungulates includes pigs, cows, goats, horses, sheep, and the like. The order Carnivora includes dogs, cats, and the like. Primates refers to mammals belonging to the order Primates, and examples of primates include the suborder Prosimii such as fox monkeys, Loris, and tree shrews, and Anthropoidea such as monkeys, apes, and humans.

“BMP inhibitor” as used herein is a compound or composition that targets BMP (bone morphogenetic protein) receptors or BMP signaling activity, including signaling initiated by BMP4. Exemplary BMP receptor inhibitors include, but are not limited to, DMH1, LDN-212854, MHL347, Dorsomorphin, LDN-193189, and LDN-213117.

As used herein, the term “growth factor” means a substance capable of stimulating cellular processes including but not limited to growth, proliferation, morphogenesis or differentiation. “Epidermal growth factor” or “EGF” is a cell signaling molecule involved in diverse cellular functions, including cell proliferation, differentiation, motility, and survival, and in tissue development. EGF is an example of a mitogenic growth factor.

As used herein, a “ROCK inhibitor” (also referred to as a “Rho-kinase inhibitor” or “rho-associated protein kinase inhibitor”) is a compound that targets rho kinase (ROCK) and inhibits the ROCK pathway. Exemplary ROCK inhibitors include, but are not limited to, AT-13148, BA-1049, BA-210, β-Elemene, Chroman 1, DJ4, GSK-576371, GSK429286A, H-1152, Y-27632, HA-1077 (fasudil), hydroxyfasudil, ibuprofen, LX-7101, netarsudil, RKI-1447, ripasudil, TCS-7001, thiazovivin, verosudil (AR-12286), Y-27632, Y-30141, Y-33075, and Y-39983. In some embodiments, the ROCK inhibitor is Y-27632.

As used herein, a “perturbagen” is a substance (e.g., chemical or biomolecular, such as a peptide) that can alter the functioning of one or more intracellular signaling pathways. In some embodiments, the perturbagen is a GSK-3 inhibitor and/or a Wnt agonist. Other pertubagens include, but are not limited to, BMP inhibitors (e.g., DMH-1) and TGF-β inhibitors (e.g., A-83-01). In some embodiments, the GSK-3 inhibitor and/or Wnt agonist is CHIR-99021 (6-((2-((4-(2,4-Dichlorophenyl)-5-(4-methyl-1H-imidazol-2-yl)pyrimidin-2-yl)amino)ethyl)amino)nicotinonitrile).

In developmental biology, “cellular differentiation” is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

A “serum-free medium” is one that contains no animal or human serum of any type. Serum-free media may be preferred to minimize possible xeno-contamination of the stem cells. A “serum replacement-free medium” is one that is free of any commercial serum replacement formulation.

As used herein, “feeder free” refers to of the absence of animal-derived cells.

The skin is composed of three layers: a) the epidermis, b) the dermis, and c) the hypodermis. The “epidermis” is the outermost layer of skin and comprises the interfollicular epidermis (IFE), which is a a stratified squamous epithelium of the skin, and hair follicles. The “dermis” is the middle layer of the skin and is composed of a network of collagenous extracellular material, fibroblasts, blood vessels, nerves, sebaceous glands, and elastic fibers. The “hypodermis” is the layer underneath the dermis and mainly comprises adipose tissue and sweat glands.

The “hair follicle” or “hair follicle mini-organ” is a peg of tissue that comprises a highly organized system of recognizably different layers arranged in concentric series. Active hair follicles extend down through the dermis, the hypodermis (a loose layer of connective tissue), and into the fat or adipose layer (Ross M H, Histology: A text and atlas, 3rd Ed., Williams and Wilkins, 1995: Chapter 14; Burkitt H G, et al, Wheater's Functional Histology, 3rd Ed., Churchill Livingstone, 1996: Chapter 9).

The “hair bulb” comprises a body of dermal cells or “dermal compartment”, also known as the “dermal papilla” cells or “DP” cells, contained in an inverted cup of epidermal cells known as the epidermal “matrix.” The hair bulb can be found at the base of an active hair follicle. Irrespective of follicle type, the germinative epidermal cells at the very base of this epidermal “matrix” produce the hair fiber, together with several supportive epidermal layers. The lowermost dermal sheath is contiguous with the papilla basal stalk, from where the sheath curves externally around all of the hair matrix epidermal layers as a thin covering of tissue. The lowermost portion of the dermal sheath then continues as a sleeve or tube for the length of the follicle (Ross M H, Histology: A text and atlas, 3rd Ed., Williams and Wilkins, 1995: Chapter 14; Burkitt H G, et al, Wheater's Functional Histology, 3rd Ed., Churchill Livingstone, 1996: Chapter 9).

As used herein, “Dermal Papilla cell” or “DP cell” refers to a specialized mesenchymal cell that regulates hair follicle development, growth, and cycling when present in the dermal papilla of a hair follicle, and is also a reservoir of multi-potent DP stem cells.

“Organoid”, as used herein, refers to a three-dimensional cell cluster or aggregate derived through culture of stem cells that has structural similarity to an organ, or part of an organ, and possesses cell types relevant to that particular organ. Organoids include miniaturized and/or simplified versions of an organ and usually replicate much of the complexity of an organ, or at least express selected aspects of it, such as producing only types of cells relevant to that particular organ. Hair follicle organoids are described herein.

As used herein, a “fibroblast” is a cell in connective tissue which produces an extracellular matrix rich in collagen and other fibers.

As used herein, “skin graft” or “replacement skin graft” refers to a patch of skin for transplantation, implantation, or other attachment, to a subject.

As used herein, a “cellular scaffold” or “tissue scaffold” refers to a material designed to cause desirable cellular interactions to contribute to the formation of new functional tissues for implantation or other medical purposes. In some embodiments, the cellular scaffold is in the form of a hydrogel, a fiber network (e.g., via electrospun mat), or a photoinitiated 3D structure. In some embodiments, the cellular scaffold comprises a biodegradable polymer.

In some embodiments, the cellular scaffold is in the form of a hydrogel. As used herein, “hydrogel” refers to a gel in which the liquid component is water. Examples of hydrogels include, but are not limited to, gelatin-based hydrogels, collagen-based hydrogels, hyaluronic-based hydrogels, polynucleotide-based hydrogels, polypeptide-based hydrogels, polysaccharide-based hydrogels, poly(ethylene glycol) (PEG)-based hydrogels, and polyacrylate-based hydrogels. See, e.g., El-Sherbiny et al., Glob Cardiol Sci Pract. 2013(3):316-342. In some embodiments, the cellular scaffold includes a cell reservoir and a guide attached to the cell reservoir comprising one or more first biodegradable polymers. First biodegradable polymers include, but are not limited to, poly(glycolic acid) (PGA), poly(lactic-co-glycolic) acid (PLGA), poly(lactic acid) (PLA), polycaprolactone (PCL), polyethylene glycol, poly(butylene succinate) (PBS), polyphosphazenes, polyanhydrides, polyphosphoesters, polyurethanes, polycarbonates, and combinations thereof.

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Or” is used in the inclusive sense, i.e., equivalent to “and/or,” unless the context requires otherwise.

“About” indicates a degree of variation that does not substantially affect the properties of the described subject matter, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed considering the number of reported significant digits and by applying ordinary rounding techniques.

### II. Exemplary Methods, Cells, and Uses Thereof

### A. Method for Producing Epithelial Stem Cells (EPSCs)

Methods of producing epithelial stem cells (EPSCs) from human induced pluripotent stem cells (hiPSCs) are provided herein.

In some embodiments, a method of producing epithelial stem cells (EPSCs) from human induced pluripotent stem cells (hiPSCs) comprises:
(a) culturing hiPSCs in a medium comprising retinoic acid (RA);
(b) culturing the cells of step (a) in a medium comprising RA, bone morphogenetic protein 4 (BMP4), and epidermal growth factor (EGF);
(c) culturing the cells of step (b) in a medium comprising a keratinocyte serum-free medium, RA, BMP4, EGF, and ROCK inhibitor; and
(d) culturing the cells of step (c) in a medium comprising a keratinocyte serum-free medium, BMP4, EGF, and Rock inhibitor, thereby producing EPSCs.

In some embodiments, one or more, or each, of the culturing steps (a), (b), (c), and (d) are conducted for a duration independently selected from about 1, 2, 3, or more days.

In some embodiments, at least one, two, or three of the culturing steps (a), (b), (c), and (d) is conducted for 2 days. In some embodiments, the culturing steps (a), (b), (c), and (d) are each conducted for 2 days.

In some embodiments, a method of producing epithelial stem cells (EPSCs) from human induced pluripotent stem cells (hiPSCs) comprises: culturing hiPSCs in a medium comprising retinoic acid (RA) for 2 days; culturing the cells of step (a) in a medium comprising RA, bone morphogenetic protein 4 (BMP4), and epidermal growth factor (EGF) for 2 days; culturing the cells of step (b) in a medium comprising RA, BMP4, EGF, and ROCK inhibitor for 2 days; and culturing the cells of step (c) in a medium comprising BMP4, EGF, and Rock inhibitor for 2 days.

In some embodiments, the medium of culturing step a does not include BMP4.

In some embodiments, the medium of culturing step (a) further comprises a keratinocyte serum-free medium and a matrix comprising a solubilized basement membrane matrix secreted by Engelbreth-Holm-Swarm (EHS) mouse sarcoma cells and human Collagen Type I (BMM/Coll1 matrix).

In some embodiments, the medium of culturing step (b) further comprises the BMM/Coll1 matrix.

In some embodiments, the medium of culturing step (c) further comprises human Collagen type I, fibronectin, and laminin-521 (Coll1/FN/LN521).

In some embodiments, the medium of culturing step (d) further comprises Coll1/FN/LN521.

In some embodiments, the medium used in culturing steps (a)-(d) is feeder-free and serum-free.

In some embodiments, a method of producing epithelial stem cells (EPSCs) from human induced pluripotent stem cells (hiPSCs) comprises: differentiating hiPSCs into EPSCs using a feeder-free and serum-free system comprising retinoic acid (RA), bone morphogenetic protein 4 (BMP4), epidermal growth factor (EGF), and ROCK inhibitor.

In some embodiments, the EPSCs express TP63 and KRT15.

In some embodiments, the method further comprises culturing the hiPSCs for one or more days with: i. a BMM/Coll1 matrix; and ii. a serum-free medium comprising basal medium, recombinant human basic fibroblast growth factor (rh bFGF), and recombinant human transforming growth factor β (rhTGFβ).

In some embodiments, the method further comprises expanding the EPSCs by maintaining the cells on a feeder-free medium.

In some embodiments, the feeder-free maintaining step comprises: (a) culturing in a feeder-free medium comprising BMP4, EGF, and ROCK inhibitor; and (b) maintaining the cells of step (a) in a feeder-free medium comprising EGF and ROCK inhibitor.

In some embodiments, culturing step (a) and maintaining step (b) are each conducted for 1, 2, 3, or more days, which may be the same or different number of days.

In some embodiments, culturing step (a) and maintaining step (b) are both conducted for 2 days.

In some embodiments, the feeder-free medium of culturing step (a) further comprises Coll1/FN/LN521 and a keratinocyte serum-free medium.

In some embodiments, the feeder-free medium of maintaining step (b) further comprises a keratinocyte serum-free medium.

In some embodiments, the expanding step is for at least 2, least 3, at least 4, at least 5, or at least 6 passages.

In some embodiments, the expanding step is for 6 passages.

In some embodiments, the method further comprises expanding the EPSCs by maintaining the cells on a feeder medium comprising mitotically inactivated 3T3-J2 mouse fibroblasts.

In some embodiments, the feeder medium further comprises ROCK inhibitor.

In some embodiments, the feeder medium further comprises Coll1/FN/LN521 and a keratinocyte growth medium. In some embodiments, the keratinocyte growth medium comprises DMEM/F12, dipeptide L-alanine-L-glutamine, keratinocyte growth medium 2 (KG2) basal medium, fetal bovine serum (FBS), adenine, an effective amount of one or more antibiotics and/or antimycotics, hydrocortisone, cholera toxin (CT), epidermal growth factor (EGF), and insulin.

In some embodiments, the feeder medium further comprises DMEM, bovine calf serum (BCS), one or more antibiotics and/or antimycotics, and L-Glutamine. In some embodiments, the one or more antibiotics and/or antimycotics comprise penicillin, streptomycin, and amphotericin B.

In some embodiments, the EPSCs have increased expression of markers TP63, KRT14, and KRT15 after/during expansion as compared to iPSC controls.

In some embodiments, the BMM/Coll1 matrix is formed by: combining BMM with DMEM/F12 to form a diluted BMM solution; adding human Collagen Type I (Coll1) solution to the diluted BMM solution to form a BMM/Coll1 matrix.

In some embodiments, the EPSC expresses CD59, CD99, LEF1, TCF4, and LGR6. In some embodiments, the EPSC expresses CD59, CD99, KRT15, KRT19, ITGA6, ITGB1, LEF1, TCF4, TP63, and LGR6. HF bulge EPSC signature genes in Wnt signaling pathway include CD59, CD99, LEF1 and TCF4, and a downstream target gene of Wnt signaling is LGR6.

In some embodiments, the method further comprises culturing the EPSCs with a perturbagen. In some embodiments, the perturbagen is a GSK-3 inhibitor and/or a Wnt agonist. In some embodiments, the GSK-3 inhibitor and/or Wnt agonist is CHIR-99021 (6-((2-((4-(2,4-Dichlorophenyl)-5-(4-methyl-1H-imidazol-2-yl)pyrimidin-2-yl)amino)ethyl)amino)nicotinonitrile).

### B. Culture Media

A culture medium used in methods described herein may contain serum. Serum obtained from any appropriate source may be used, including fetal bovine serum (FBS), goat serum or human serum. In some embodiments, human serum is used. Serum may be used at between about 1% and about 30% by volume of the medium, according to conventional techniques.

In other embodiments, a culture medium of the invention may contain a serum replacement. Various different serum replacement formulations are commercially available and are known to the skilled person. Where a serum replacement is used, it may be used at between about 1% and about 30% by volume of the medium, according to conventional techniques.

In other embodiments, a culture medium of the invention may be serum-free and/or serum replacement-free. A “serum-free medium” is one that contains no animal serum of any type. Serum-free media may be preferred to avoid possible xeno-contamination of the stem cells. A “serum replacement-free medium” is one that has not been supplemented with any commercial serum replacement formulation.

The medium may also include a feeder support system supplemented with fetal bovine serum or serum replacer, or the medium may be a feeder-free system optionally supplemented with defined culture media, such as mTeSR™1 and/or TeSR™8. As used herein, “feeder free” refers to a minimal use of animal-derived cells and proteins.

### C. Cells

Also provided herein is an EPSC (e.g., mammalian EPSC, such as a human EPSC) derived from an iPSC (i.e., iPSC-EPSC), e.g., produced according to the methods described herein. An EPSC derived from a human iPSC is referred to as an hiPSC-EPSC. In some embodiments, the iPSC-EPSC differs from naturally occurring EPSCs in at least one aspect. For example, one or more markers may be differentially expressed in the iPSC-EPSC produced according to the methods described herein compared to naturally occurring EPSCs (e.g., CD59, CD200, KRT15, KRT19, DKK3, FZD1, FZD2, CTNNB1, LEF1, LGR6, TGFB2, ITGA6, ITGB1, and DCN).

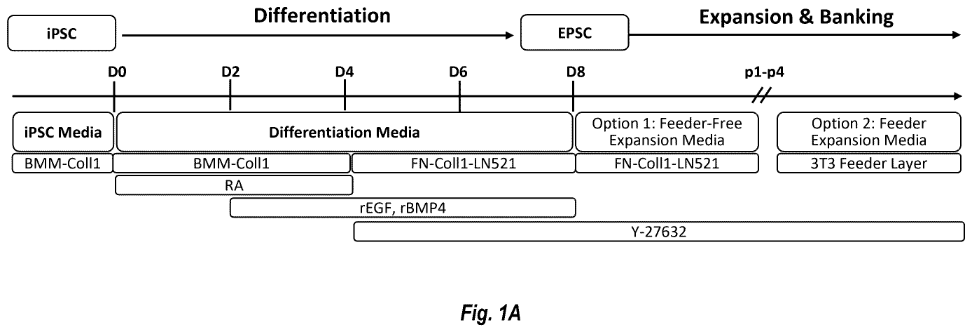

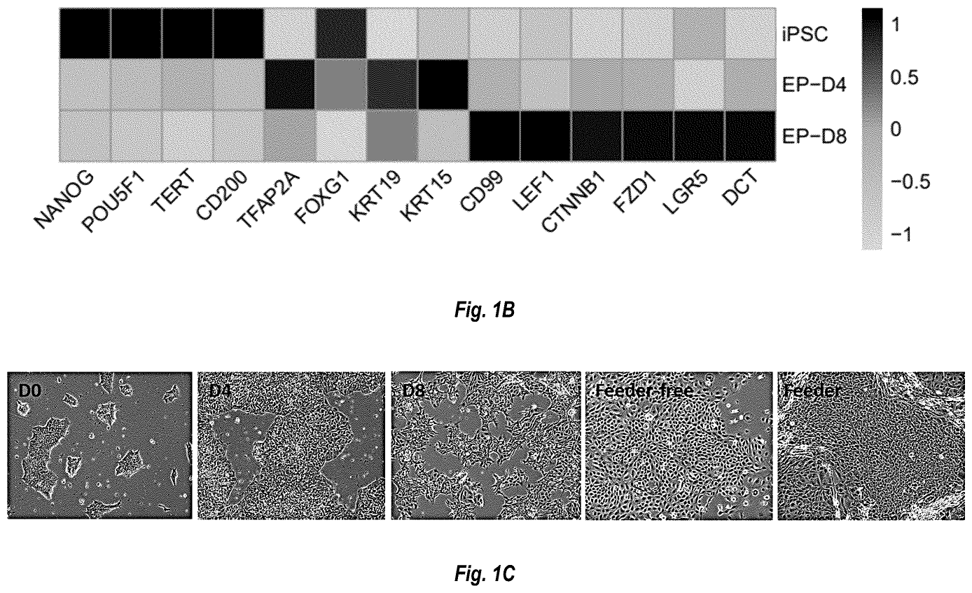

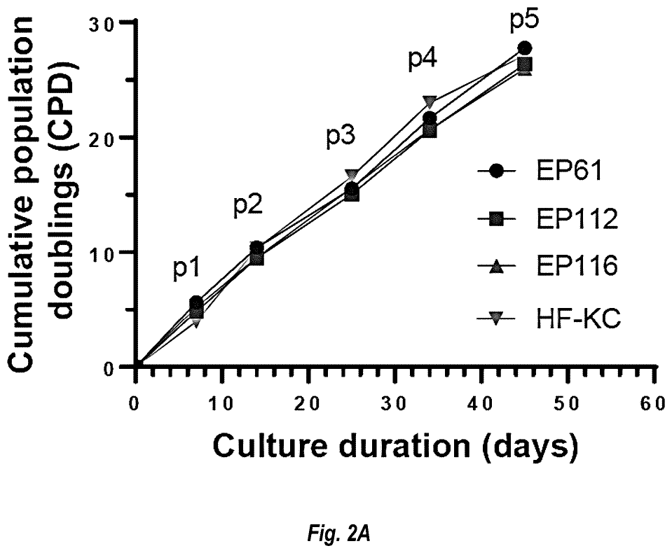

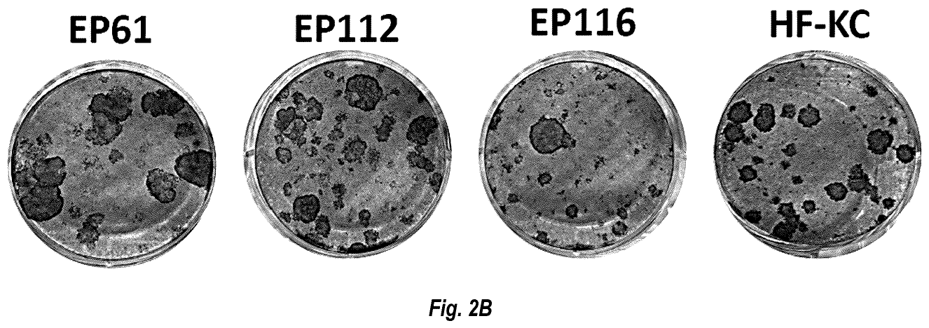

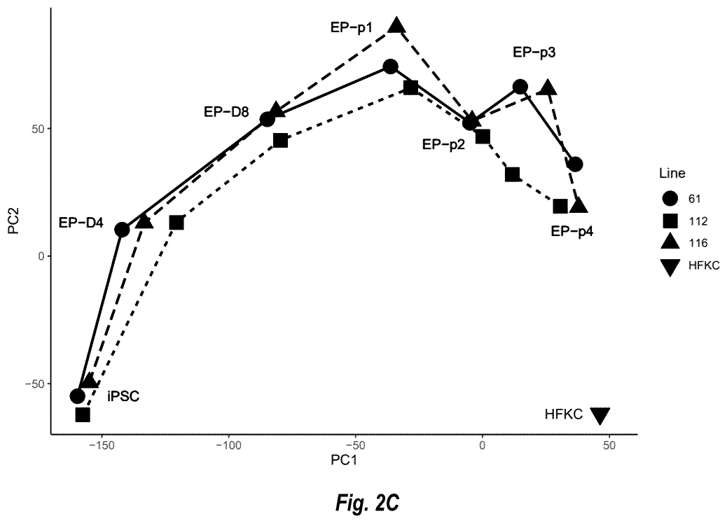

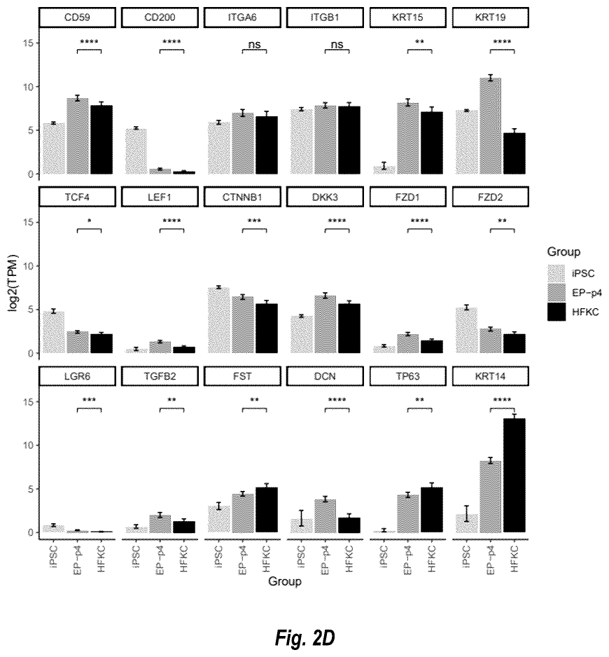

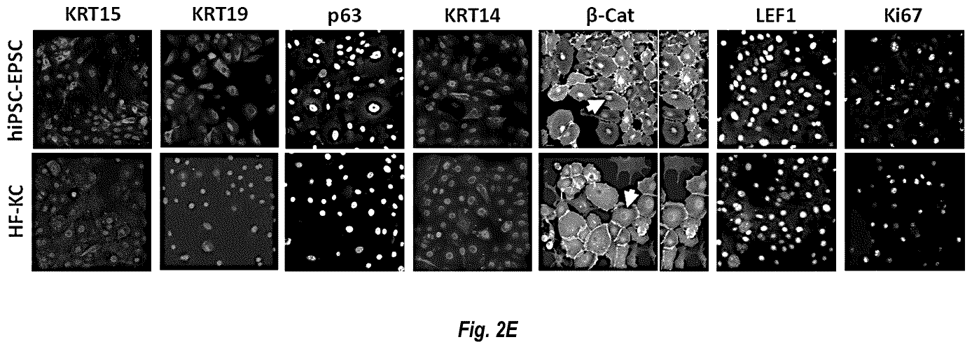

FIG. 2D shows comparative EPSC marker expression between iPSC, hiPSC-derived EP, and an equivalent of naturally occurring human bulge IF-EPSC (specifically, bulge HF-derived keratinocytes, i.e., cells expanded from the bulge of adult hair follicle). FIG. 2D shows that when compared to HF-KC, hPSC-EPSC express elevated levels of EPSC markers CD59, CD200, KRT15, KRT19, DKK3, FZD1, FZD2, CTNNB1, LEF1, LGR6, TGFB2, and DCN; similar levels of ITGA6 and ITGB1; and reduced levels of KRT14 and TP63. In some embodiments, the iPSC-EPSCs have increased expression of markers TP63, KRT5, KRT14, and KRT15 after/during expansion as compared to iPSC controls. See also, e.g., the expression of markers shown in FIG. 1B and FIG. 2D Alternatively or in addition thereto, the iPSC-EPSC may have a greater capacity for self-renewal and/or a greater capacity to form organoids than naturally occurring EPSCs. Natural and iPSC-EPSC have similar proliferation dynamics and colony formation.

### D. Organoid Production

In some embodiments, an organoid, such as a folliculogenic organoid, is produced using the EPSCs produced by one of the disclosed methods. Organoid formation may comprise culturing the EPSCs in the presence of an additional cell type, such as dermal papilla cells. The culturing may occur within a scaffold, e.g., a biodegradable scaffold or a hydrogel scaffold. Exemplary procedures for formation and use of organoids are described, e.g., in US 2022/0105247 A1, published Apr. 7, 2022. In some embodiments, organoid formation may comprise forced aggregation of cells, e.g., in an ultralow attachment environment or a droplet of a solubilized basement membrane preparation extracted from the Engelbreth-Holm-Swarm (EHS) mouse sarcoma (e.g., a MATRIGEL® (CORNING®) droplet). See, e.g., Kretzschmar et al., Dev Cell 38(6):590-600 (2016); Sato et al., Nature 459(7244):262-5 (2009).

In some embodiments hiPSC-EPSC are combined with dermal papilla (DP) cells and optionally hair follicle keratinocytes (HF-KC), which are collectively capable of self-assembling into elongated organoids resembling natural HF through the stages of aggregation, polarization and elongation. In some embodiments, a GSK-3 inhibitor, such as CHIR-99021, is added to the EPSC and organoid culture.

In some embodiments, dermal layers (or skin equivalents) are generated using EPSCs described herein in combination with foreskin derived fibroblasts (HFF). In some embodiments, the HFFs are cultured for 14 days to obtain the dermal layer. In some embodiments, hiPSC-EPSCs are seeded onto the dermal layer, and tissue is generated. In some embodiments, the tissue generated by hiPSC-EPSC demonstrated stratified epithelia with cellular architecture and marker expression similar to that generated by control NHK. In some embodiments, the tissues generated contained epidermal differentiation marker K10 (KRT10) in the suprabasal layer, epidermal differentiation marker K14 (KRT14) in the basal and central layer of stratified epithelia, and proliferation marker Ki67 in the basal layer of the stratified tissue.

### E. Applications

### 1. Methods of Producing or Growing Hair Follicles

In some embodiments, the EPSCs may be used to obtain hair follicles and/or interfollicular epidermis.

In some embodiments, a method of obtaining hair follicles and/or interfollicular epidermis comprises culturing the EPSC under conditions to obtain hair follicles and/or interfollicular epidermis.

In some embodiments, a method of producing hair follicles comprises culturing the EPSCs with fibroblasts and/or dermal papilla cells to obtain hair follicles. In some embodiments, a method of growing a hair follicle comprises: loading one or more EPSCs into a cellular scaffold; and implanting the cellular scaffold into dermal tissue. In some embodiments, the cellular scaffold comprises a hydrogel. In some embodiments, the cellular scaffold comprises: a cell reservoir; and a guide attached to the cell reservoir comprising one or more first biodegradable polymers. Exemplary cellular scaffolds are described, e.g., in US 2022/0105247 A1, published Apr. 7, 2022.

In some embodiments, a method of growing a hair follicle comprises implanting the organoid produced using the EPSCs into dermal tissue. The organoid may be contained within a cellular scaffold. In some embodiments, the method further comprises piercing the dermal tissue with a needle prior to implanting the organoid or cellular scaffold.

In some embodiments, the method of growing a hair follicle comprises combining the EPSCs with dermal cells, and implanting the combined EPSCs and dermal cells into a subject below the epidermis (e.g., by subcutaneous injection), wherein the hair follicle is generated in the subject from the combination.

### 2. Methods of Producing Skin Grafts

In some embodiments, the EPSCs may be used for replacement skin grafts.

In some embodiments, a method of producing a skin graft comprises culturing the EPSCs under conditions to produce a skin graft. Methods of generating skin grafts from EPSC are generally known. For example, Zang et al., Nat Commun. 5:3071 (2014), discloses generation of skin grafts from EPSCs. As another example, Morris et al., Nat Biotechnol. 22(4):411-7 (2004), and Ito et al., Nature 447(7142):316-20 (2007), discloses generation of skin grafts from naturally occurring bulge EPSC.

In some embodiments, the iPSC-EPSCs are cultured with a dermal component (e.g., dermal fibroblasts and a collagen substrate) to obtain a skin graft (i.e., a cultured skin graft). In some embodiments, iPSC-EPSCs are delivered (e.g., sprayed) onto a shallow wound bed to repopulate the epidermal layer and thus form a skin graft on site. The iPSC-EPSCs may be expanded in culture before such delivery.

### III. Additional Embodiments

The following are additional embodiments encompassed by the disclosure.

Embodiment 1 is a method of producing epithelial stem cells (EPSCs) from human induced pluripotent stem cells (hiPSCs), wherein the method comprises:
a. culturing hiPSCs in a medium comprising retinoic acid (RA);
b. culturing the cells of step 2a in a medium comprising RA, bone morphogenetic protein 4 (BMP4), and epidermal growth factor (EGF);
c. culturing the cells of step 2b in a medium comprising a keratinocyte serum-free medium, RA, BMP4, EGF, and ROCK inhibitor; and
d. culturing the cells of step 2c in a medium comprising a keratinocyte serum-free medium, BMP4, EGF, and Rock inhibitor, thereby producing EPSCs.

Embodiment 2 is the method of embodiment 1, wherein the culturing steps a, b, c, and d are conducted for a duration independently selected from about 1, 2, 3, or more days.

Embodiment 3 is the method of embodiment 2, wherein the culturing steps a, b, c, and d are each conducted for 2 days.

Embodiment 4 is a method of producing epithelial stem cells (EPSCs) from human induced pluripotent stem cells (hiPSCs), wherein the method comprises:
a. culturing hiPSCs in a medium comprising retinoic acid (RA) for 2 days;
b. culturing the cells of step 2a in a medium comprising RA, bone morphogenetic protein 4 (BMP4), and epidermal growth factor (EGF) for 2 days;
c. culturing the cells of step 2b in a medium comprising RA, BMP4, EGF, and ROCK inhibitor for 2 days; and
d. culturing the cells of step 2c in a medium comprising BMP4, EGF, and Rock inhibitor for 2 days.

Embodiment 5 is the method of any one of the preceding embodiments, wherein the medium of culturing step a does not include BMP4.

Embodiment 6 is the method of any one of the preceding embodiments, wherein the medium of culturing step a further comprises a keratinocyte serum-free medium and a matrix comprising a solubilized basement membrane matrix secreted by Engelbreth-Holm-Swarm (EHS) mouse sarcoma cells and human Collagen Type I (BMM/Coll1 matrix).

Embodiment 7 is the method of any one of the preceding embodiments, wherein the medium of culturing step b further comprises the BMM/Coll1 matrix.

Embodiment 8 is the method of any one of the preceding embodiments, wherein the medium of culturing step c further comprises human Collagen type I, fibronectin, and laminin-521 (Coll1/FN/LN521).

Embodiment 9 is the method of any one of the preceding embodiments, wherein the medium of culturing step d further comprises Coll1/FN/LN521.

Embodiment 10 is the method of any one of the preceding embodiments, wherein the medium used in culturing steps a-d is feeder-free and serum-free.

Embodiment 11 is a method of producing epithelial stem cells (EPSCs) from human induced pluripotent stem cells (hiPSCs) comprising:
differentiating hiPSCs into EPSCs using a feeder-free and serum-free system comprising retinoic acid (RA), bone morphogenetic protein 4 (BMP4), epidermal growth factor (EGF), and ROCK inhibitor.

Embodiment 12 is the method of any one of the preceding embodiments, wherein the EPSCs express TP63, KRT5, KRT14, and KRT15.

Embodiment 13 is the method of any one of the preceding embodiments, further comprising culturing the hiPSCs for one or more days with:
i. a BMM/Coll1 matrix; and
ii. a serum-free medium comprising basal medium, recombinant human basic fibroblast growth factor (rh bFGF), and recombinant human transforming growth factor β (rh TGFβ).

Embodiment 14 is the method of any one of the preceding embodiments, wherein the method further comprises expanding the EPSCs by maintaining the cells on a feeder-free medium.

Embodiment 15 is the method of embodiment 14, wherein the feeder-free maintaining step comprises:
i. culturing in a first feeder-free medium comprising BMP4, EGF, and ROCK inhibitor; and
ii. maintaining the cells of step i. in a second feeder-free medium comprising EGF and ROCK inhibitor, optionally wherein the second feeder-free medium does not comprise BMP4.

Embodiment 16 is the method of embodiment 15, wherein culturing step i. and maintaining step ii. are each conducted for 1, 2, 3, or more days, which may be the same or different number of days.

Embodiment 17 is the method of embodiment 16, wherein culturing step i. and maintaining step ii. are both conducted for 2 days.

Embodiment 18 is the method of any one of embodiments 15 to 17, wherein the feeder-free medium of culturing step i. further comprises Coll1/FN/LN521 and a keratinocyte serum-free medium.

Embodiment 19 is the method of any one of embodiments 15 to 18, wherein the feeder-free medium of maintaining step ii. further comprises a keratinocyte serum-free medium.

Embodiment 20 is the method of any one of embodiments 14 to 19, wherein the expanding step is for at least 2, least 3, at least 4, at least 5, or at least 6 passages.

Embodiment 21 is the method of the immediately preceding embodiment, wherein the expanding step is for 6 passages.

Embodiment 22 is the method of any one of embodiments 1-13, wherein the method further comprises expanding the EPSCs by maintaining the cells on a feeder medium comprising mitotically inactivated 3T3-J2 mouse fibroblasts.

Embodiment 23 is the method of the immediately preceding embodiment, wherein feeder medium further comprises ROCK inhibitor.

Embodiment 24 is the method of embodiment 22 or embodiment 23, wherein the feeder medium further comprises Coll1/FN/LN521 and a keratinocyte growth medium.

Embodiment 25 is the method of the immediately preceding embodiment, wherein the keratinocyte growth medium comprises DMEM/F12, dipeptide L-alanine-L-glutamine, keratinocyte growth medium 2 (KG2) basal medium, fetal bovine serum (FBS), adenine, an effective amount of one or more antibiotics and/or antimycotics, hydrocortisone, cholera toxin (CT), epidermal growth factor (EGF), and insulin.

Embodiment 26 is the method of any one of embodiments 22 to 25, wherein the feeder medium further comprises DMEM, bovine calf serum (BCS), one or more antibiotics and/or antimycotics, and L-Glutamine.

Embodiment 27 is the method of embodiment 25 or embodiment 26, wherein the one or more antibiotics and/or antimycotics comprise penicillin, streptomycin, and amphotericin B.

Embodiment 28 is the method of any one of the preceding embodiments, wherein the EPSCs have increased expression of markers TP63, KRT5, KRT14, and KRT15 after/during expansion as compared to iPSC controls.

Embodiment 29 is the method of any one of embodiments 6-10 and 13-28, wherein BMM/Coll1 matrix is formed by:
a. combining BMM with DMEM/F12 to form a diluted BMM solution;
b. adding human Collagen Type I (Coll1) solution to the diluted BMM solution to form a BMM/Coll1 matrix.

Embodiment 30 is the method of any one of the proceeding embodiments, wherein the method further comprises culturing the EPSCs with a perturbagen.

Embodiment 31 is the method of embodiment 30, wherein the perturbagen is a GSK-3 inhibitor and/or Wnt agonist.

Embodiment 32 is the method of embodiment 31, wherein the GSK-3 inhibitor and/or Wnt agonist is CHIR-99021.

Embodiment 33 is the method of any one of the preceding embodiments, wherein the EPSC expresses at least two, three, four, or each of CD59, CD99, LEF1, TCF4, and LGR6.

Embodiment 34 is the method of any one of the preceding embodiments, wherein the EPSC expresses at least two, three, four, five, six, seven, eight, nine, or each of CD59, CD99, KRT15, KRT19, ITGA6, ITGB1, LEF1, TCF4, TP63, and LGR6.

Embodiment 35 is an epithelial stem cell (EPSC) produced by the method of any one of the preceding embodiments.

Embodiment 36 is the EPSC of the immediately preceding embodiment, wherein the EPSC expresses one or more of CD59, CD200, KRT19, DKK3, FZD1, FZD2, CTNNB1, LEF1, LGR6, TGFB2, and DCN at an increased level relative to a naturally occurring EPSC, and/or one or more of ITGA6, ITGB1, K14 and TP63 at a decreased level relative to a naturally occurring EPSC.

Embodiment 37 is an EPSC differentiated from an induced pluripotent stem cell, wherein the EPSC expresses one or more of CD59, CD200, KRT19, DKK3, FZD1, FZD2, CTNNB1, LEF1, LGR6, TGFB2, and DCN at an increased level relative to a naturally occurring EPSC, and/or one or more of ITGA6, ITGB1, K14 and TP63 at a decreased level relative to a naturally occurring EPSC.

Embodiment 38 is the EPSC of embodiment 36 or 37, wherein the EPSC expresses two, three, four, five, six, seven, eight, nine, ten, or each of CD59, CD200, KRT19, DKK3, FZD1, FZD2, CTNNB1, LEF1, LGR6, TGFB2, and DCN at an increased level relative to a naturally occurring EPSC.

Embodiment 39 is the EPSC of any one of embodiments 36-38, wherein the EPSC expresses two, three, or each of ITGA6, ITGB1, K14 and TP63 at a decreased level relative to a naturally occurring EPSC.

Embodiment 40 is the EPSC of any one of embodiments 36-39, wherein the naturally occurring EPSC is a bulge hair follicle-derived ketatinocyte.

Embodiment 41 is the EPSC of any one of embodiments 36-40, which is a human EPSC.

Embodiment 42 is a method of culturing the EPSC of any one of embodiments 35-41 under conditions to obtain hair follicles and/or interfollicular epidermis.

Embodiment 43 is an organoid produced using the EPSCs of any one of embodiments 35-41.

Embodiment 44 is the organoid of the immediately preceding embodiment, wherein the organoid is a folliculogenic organoid.

Embodiment 45 is a method of producing hair follicles comprising culturing the EPSCs of any one of embodiments 35-41 with fibroblasts and/or dermal papilla cells to obtain hair follicles.

Embodiment 46 is a method of producing a skin graft comprising culturing the EPSCs of any one of embodiments 35-41 under conditions to produce a skin graft.

Embodiment 47 is a method of producing a skin graft comprising culturing the EPSCs of any one of embodiments 35-41 under conditions to produce a cultured epidermal graft.

Embodiment 48 is the use of the EPSCs of any one of embodiments 35-41 to obtain hair follicles and/or interfollicular epidermis.

Embodiment 49 is the use of the EPSCs of any one of embodiments 35-41 for a replacement skin graft.

Embodiment 50 is the method of embodiment 47 or the use of embodiment 49, comprising delivering the EPSCs onto a wound bed, optionally wherein delivering comprises spraying.

Embodiment 51 is the method or use of embodiment 49 or 50, wherein the EPSCs are expanded in culture, optionally wherein the expansion occurs before delivering the EPSCs onto a wound bed.

Embodiment 52 is the method or use of embodiment 51, wherein the EPSCs are cultured with a dermal component, optionally wherein the dermal component comprises dermal fibroblasts and/or a collagen substrate.

Embodiment 53 is a method of growing a hair follicle, comprising:
a. loading one or more EPSCs according to any one of embodiments 35-41 into a cellular scaffold; and
b. implanting the cellular scaffold into dermal tissue.

Embodiment 54 is the method of embodiment 53, wherein the cellular scaffold is in the form of a hydrogel, a fiber network, or a photoinitiated 3D structure.

Embodiment 55 is the method of embodiment 53 or embodiment 54, wherein the cellular scaffold comprises a biodegradable polymer.

Embodiment 56 is the method of any one of embodiments 53-55, wherein the cellular scaffold comprises:
i. a cell reservoir; and
ii. a guide attached to the cell reservoir comprising one or more first biodegradable polymers.

Embodiment 57 is a method of growing a hair follicle, comprising implanting the organoid of embodiment 43 or embodiment 44 into dermal tissue.

Embodiment 58 is the method of the immediately preceding embodiment, further comprising piercing the dermal tissue with a needle prior to implanting the organoid.

Embodiment 59 is the method of embodiment 57 or embodiment 58, wherein a cellular scaffold containing the organoid is implanted into the dermal tissue.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define the scope of the invention and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website or the like are published at different times, the version most recently published at the effective filing date of the application is meant, unless otherwise indicated. To the extent that any material incorporated by reference is inconsistent with the express content of this disclosure, the express content controls.

### EXAMPLES

### Materials and Methods

Generation of hiPSC-EPSC: Prior to differentiation, hiPSC were cultured on a thin layer of solubilized basement membrane extract (BME) (1:60 dilution in DMEM for 30 min, CORNING®) in mTeSR1 medium (STEMCELL Tech). For differentiation, 80% confluent hiPSCs were dissociated into small clusters using ReLeSR™ (STEMCELL Tech) and re-plated onto plates coated with BMM (1:60 dilution in DMEM, CORNING®) mixed with hColl1 (0.25 μg/cm², Stem Cell Tech) in mTeSR™1 medium (STEMCELL Tech). After 24 hours, the media was changed to the differentiation medium, and cells were grown to 90% confluency for 4 days. On day 4, cells were dissociated to using ACCUTASE™ (STEMCELL Tech) and replated onto plates coated with triple matrix (TM) consist of hColl1 (0.25 μg/cm², Stem Cell Tech), fibronectin (0.25 μg/cm², Sigma), and Laminin 521 (0.2 μg/cm², CORNING®) in differentiation media supplemented with Y-27632 (10 μM, Sigma). Cells were grown for additional 4 days until epithelial cells were observed on day 8. For differentiation to the epithelial lineage, the following molecules were used: days 0-2, all-trans RA (1 M, Sigma); days 2-6, all-trans RA (1 μM, Sigma), EGF (20 ng/ml, Sigma) and BMP4 (25 ng/ml, R&D systems); days 6-8, EGF (20 ng/ml, Sigma) and BMP4 (25 ng/ml, R&D systems); and Defined Keratinocyte-SFM (Invitrogen). On day 8, cells were dissociated using ACCUTASE™ (STEMCELL Tech) and re-plated on either TM substrate for feeder-free expansion or on a feeder layer of Mitomycin-C-treated 3T3-J2 fibroblasts (MmC-3T3) for feeder-based expansion. For feeder-free expansion, Keratinocyte-SFM (Invitrogen) supplemented with Y-27632 (10 μM, Sigma) was used. For feeder-based expansion, 3:1 mixture of DMEM-F12 (GIBCO®) and KG2 basal medium (PromoCell) with 5% FBS, 0.18 mM adenine, 5 μg/ml insulin, 0.5 g/ml hydrocortisone, 0.1 nM cholera toxin, 10 ng/ml EGF, and 10 μM Y-27632 (all Sigma) was used.

3D human skin equivalent assay: 3D co-culture, full thickness skin model system was used as described by the manufacturer (CELLnTECH). Briefly, human dermal fibroblasts (CnT-HDF) were cultured in CnT-PR-F medium then seeded onto 0.4 um pore PET size insert placed within a 12 well plate (TPP #92012) and grown for two additional weeks in CnT-PR-F medium. Human primary keratinocytes (CnT-HPEK) or hiPSC-EPSC were grown in CnT-PR medium then seeded on top of the HDFs at 240,000 cells per insert and grown for 2 additional days submerged in CnT-PR medium. After three days, a sterilized-plate spacer (#CnT-SP) is placed on top of a 12 well plate and the inserts were placed on top of the spacers. CnT-PR-FTAL medium is added outside the insert into the well at equal level with the insert membrane. The air-lifted model was cultured for 12 additional days prior to fixation and immunohistochemical analysis.

Hair follicle organoid assay: After harvesting and counting the appropriate cell types needed to form organoids, each cell type was resuspended at a density of 150k cells/mL in Organoid Medium, composed of 50% of AmnioMAX™-II (ThermoFisher) and 50% of fully supplemented KG2 (PromoCell). The two cell types were then combined in a 15 mL tube at a 1:1 ratio. The cell suspension was dispensed into ultra-low attachment 384-well plate (CORNING®) by using an electric repeating pipet (Integra 125) and centrifuged at 1000 g for 5 minutes. After two days, the organoid medium was changed to Organoid media supplemented with 2% of solubilized basement membrane extract (BME, CORNING®) with or without 2.5 μm CHIR99021. Subsequently, media change was performed every 3 days followed by a full-plate scan with automated imaging system (Cytation 5, Bio-Tek). The images acquired were subjected to automated analysis to measure the roundness score (also known as elongation score) by using the Gen5™ Data Analysis Software. Based on software segmentation score, we assigned a range of values to the organoids, which are defined as aggregated if the roundness score was between 1 and 0.8, polarized between 0.8 and 0.6 and elongated when the roundness score was below 0.6.

Immunocytochemistry: Cells were cultured and imaged in 96 well plates. Briefly, media was aspirated, and wells rinsed once with PBS for 2 minutes. Cells were fixed with 4% formaldehyde for 10 minutes at RT, followed by two washes with PBS. Cells were blocked and permeabilized with PBSAT (0.5% Triton X-100 and 2% BSA diluted in PBS) for 1 hour and incubated overnight with the primary antibodies (Table 1). The cells were washed three times with PBSAT and then incubated with the corresponding secondary antibody in blocking solution for 1 hour at RT. After washing 3 times with PBSAT, nuclei were stained with DAPI (diluted 1:000 in PBSAT) for 10 minutes and washed twice with PBS and kept in PBS at 4° C. until imaged. Images were acquired using Zeiss LSM 800 confocal microscope or EVOS M7000 Imaging System.

ALU qPCR: Total DNA was extracted from isolated human EP-mouse chimeric patches and human control IPSCs using the NucleoSping DNA RapidLyse (Macherey-Nagel). DNA quantity was measured using a Qubit™ 4 Fluorometer (ThermoFisher). A standard curve established from an 8-step serial dilution of DNA extracted from a known cell count of hIPSCs was run in conjunction with the patch DNA samples. All samples were run as 3 technical replicates. qPCR was performed with the Fast SYBR® Green Master Mix (Invitrogen) according to the manufacturer's recommendation using the QuantStudio™ 5 (ThermoFisher.) The Alu-Yb8 primer sequences are F: 5-CGAGGCGGGTGGATCATGAGGT-3 and R: 5-TCTGTCGCCCAGGCCGGACT-3. The ThermoFisher app was used to generate a standard curve and derive the ng of human DNA from the Cq values in the patch samples. The average ng of human DNA per sample (avgDNA) was calculated from the three technical replicates of each sample. The number of human cells in the patch was estimated as avgDNA×# of human cells in Standard #1/ng of DNA in Standard #1/the volume of the DNA in reaction×the volume of total DNA per sample.

Bulk RNA-sequencing: RNA was extracted from each sample and quantified with Qubit. cDNA libraries were generated using the NEB library kit. All libraries were then quantified, pooled, and sequenced on Illumina platforms. Reads were first analyzed by fastQC and then trimmed for low-quality bases with Trimmomatic. Clean reads were then aligned to the human reference genome (GRCh38) with Ensembl gene annotation using STAR software. Transcripts per million (TPM) estimates were calculated using RSEM (RNA-Seq by Expectation-Maximization) and were used for visualization.

Animals: Athymic Nude-Foxn1^{nu} mice were obtained from Envigo or Charles River Laboratories. All animal procedures were performed in accordance with the PHS Policy on Humane Care and Use of Laboratory Animals and with IACUC approval.

Hair follicle reconstitution patch assay: For neonatal mouse hair follicle progenitor cells, truncal skin was removed from 1- to 2-day-old C57BL/6 pups and rinsed in Ca²⁺ and Mg²⁺ free PBS. The skin was incubated in 0.2% dispase overnight at 4° C. to separate epidermis from dermis. The dermis was then digested with 0.2% collagenase (Sigma-Aldrich, St. Louis) at 37° C. for 40-60 minutes, while the epidermis was digested with 0.25% trypsin/EDTA for 10-20 minutes at 37° C. Single-cell suspensions were strained through 40 μm filters and pelleted at 300 g. Unless otherwise stated for each intracutaneous injection, 1×10⁶ dermal cells (mouse dermal cells, human dermal papilla cells or iPSC-derived DP cells) and 1×10⁶ single epidermal cell were mixed and resuspended in a total volume of 100 μl of DMEM-F12 medium and injected (28G needle) into the hypodermis of the mouse skin, forming a bleb as previously described. After recovery from anesthesia (Isofluorane), mice were kept under normal husbandry conditions. The animals were monitored on a weekly basis, and the darkening of the skin at the site of the injection could be visible in correspondence with the control condition (mouse dermal and epithelial cells) two weeks after. The skin sites that had received the injected cells referred to as “patch” were harvested 3 weeks later and imaged under stereomicroscope.

Histology and Immunohistochemistry: After 3 weeks, mice were sacrificed, and hair patches were excised and fixed in 4% formaldehyde, processed for formalin embedding and sectioned. Some sections were analyzed by hematoxylin/eosin (H&E) staining, and the remaining sections were used for immunofluorescence analysis. For immunofluorescence, slides were deparaffinized and incubated with a blocking solution (1% gelatin from cold water fish skin dissolved in PBS with 0.5% Triton X-100) for 1 hour. The slides were incubated overnight at 4° C. with primary antibodies (Table 1). The slides were washed three times with PBS-0.5% Triton X-100 and incubated with the corresponding secondary conjugated antibody in blocking solution for 2 hours at room temperature. After washing out the secondary antibody, slides were mounted with ProLong® Diamond Antifade Mountant with DAPI (for nuclei staining). Sections were imaged with an Apotome microscope (Zeiss).

Statistical Analysis: One-way or two-way ANOVA tests, followed ad hoc by multiple comparison tests, were used to compare groups and Student's t-test to compare means of two groups as specified in each graph. GraphPad Prism was used for all of the analyses unless otherwise specified.

TABLE 1
Antibodies used for immunofluorescent analysis

| Antibody | Vendor | Catalog # | Dilution |
|---|---|---|---|
| Keratin 19 (KRT19) | SC Biotech | SC-6278 | 1:200 |
| Keratin 15 (KRT15) | Epredia | MS-1068-PO | 1:200 |
| Keratin 14 (KRT14) | Invitrogen | MA1006323 | 1:200 |
| B-Catenin | Invitrogen | PA5-19469 | 1:200 |
| LEF1 | Abcam | AB137872 | 1:200 |
| P63 | R&D Systems | AF1917 | 1:200 |
| KI67 | Invitrogen | PA5-19462 | 1:200 |
| Ku80 | Cell Signaling | 2180 | 1:400 |
| Donkey anti-goat Alexa 555 | Life Technologies | A21433 | 1:500 |
| Donkey anti-rabbit Alexa 647 | Invitrogen | A32795 | 1:500 |
| Goat anti-mouse Alexa 555 | Life Technologies | A21424 | 1:500 |
| Donkey anti-mouse Alexa 647 | Life Technologies | A31571 | 1:500 |
| Donkey anti-rabbit Alexa 647 | Invitrogen | A32795 | 1:500 |
| Donkey anti-mouse Alexa 647 | Life Technologies | A31571 | 1:500 |
| Goat anti-mouse Alexa 555 | Life Technologies | A21424 | 1:500 |

### Example 1: Generation of EPSC from hiPSC

A schematic representation of the differentiation protocol and the major regulatory factors administered at each stage are depicted in FIG. 1A. The effects of retinoic acid (RA), bone morphogenetic protein-4 (BMP4), and epidermal growth factor (EGF) on the differentiation of pluripotent stem cells into keratinocyte progenitors in vitro has been previously described. See Metallo et al., Stem Cells. 2008 February, 26(2):372-80, Epub 2007 Oct. 25; Itoh et al., Proc Natl Acad Sci USA. 2011, 108(21):8797-802; Yang et al., Nat Commun. 2014, 5:3071; Ibrahim et al., Curr Protoc Stem Cell Biol. 2020 September, 54(1):e119. In this protocol, the differentiation of hiPSCs cultivated as a monolayer on a chemically defined substrates in a serum-free media was directly induced. RA was used to induce ectodermal commitment of iPSCs, which were then differentiated in the presence of BMP4, EGF and Rho-Kinase Inhibitor (Y27632) into EPSCs. See further details in the Materials & Methods section above. As demonstrated in FIG. 1B, temporal gene expression of the HF bulge EPSC markers CD200, CD99, LGR5, and DCT (Ohyama et al., J Clin Invest. 2006 January, 116(1):249-60; Choi2016; Takahashi R et al., J Invest Dermatol. 2020 April, 140(4):764-773) was monitored. Gene expression analysis indicated that CD200 was strongly expressed in all undifferentiated hiPSCs and was significantly downregulated in response to RA treatment along with other pluripotency markers, NANOG, OCT3/4, and TERT. TFAP2A upregulation was observed on day 4 of differentiation, consistent a transition toward a non-neural ectoderm intermediate (Tchieu J et al., Cell Stem Cell. 2017 September, 21(3):399-410.e7). In contrast, downregulation of the neural ectoderm marker FOXG1 occurred on day 4, suggesting minimal contamination with off-target ectoderm lineages (Tchieu J 2017). The expression of CD99, LGR5, and DCT was upregulated on day 8 of differentiation following increased expression of KRT19 and KRT15. Importantly, increased expression of HF bulge EPSC markers correlated with the activation of genes associated with the Wnt signaling pathway, including LEF1, CTNNB1 (β-Cat), and FZD1. The Wnt signaling pathway is responsible for HF morphogenesis, and the activation of this pathway depends on the translocation of β-catenin from the cytoplasm to the nucleus and on the interactions with Lef1, which eventually regulate the transcription of downstream target genes. Aoki et al., Proc Natl Acad Sci USA. 1999 Jan. 5, 96(1):139-44; Zhang et al., Int J Med Sci. 2013, 10(6):738-746. Generated EPSC were collected on day 8 of differentiation, expanded for multiple passages, and banked for future functional studies. Morphological changes during the EPSC differentiation (D0-D8) and the expansion process are shown in FIG. 1C.

### Example 2: Characterization of hiPSC-EPSC In Vitro

To confirm the reproducibility of the differentiation protocol, the EPSC differentiation procedure was applied to multiple hiPSC lines. Cumulative population doublings (CPD) were calculated based on the cell counts obtained during sequential passaging of hiPSC-EPSC derived from three hiPSC-EPSCs lines, EP61, EP112 and EP116 (FIG. 2A). All three hiPSC-EPSC lines showed similar population doubling times comparable to that of control keratinocytes derived from adult HFs (HF-KCs). When plated for the colony formation assay (CFA) after 3 weeks in culture (p3), hiPSC-EPSCs showed similar colony forming efficiency to control HF-KCs (FIG. 2B). Consistent with the similar growth kinetics observed, EP61, EP112, and EP116 followed similar differentiation trajectories, analyzed by principal component analysis, with passage 4 samples from each line clustering most closely with control HF-KCs (FIG. 2C). Bulk RNA-seq analysis confirmed that hiPSC-EPSCs express signature genes of human bulge EPSC, including CD59, CD200, ITGA6, ITGB1, KRT15, KRT19, KRT15, TCF4, LEF1, CTNNB1, DKK3, FZD1, FZD2, LGR6, TGFB2, FST, DCN, and TP63 (Ohyama et al., J Clin Invest. 2006 January, 116(1):249-60; Snippert et al., Science. 2010 Mar. 12, 327(5971):1385-9; Nguyen et al., Nat Genet. 2009 October, 41(10):1068-75, Epub 2009 Aug. 30; Romano et al., Development. 2010, 137(9):1431-9, erratum in: Development. 2010 May, 137(10):1775; Miyachi et al., J Dermatol. 2018 December, 45(12):1403-1410, Epub 2018 Oct. 15), while the expression of the terminally differentiated keratinocyte marker KRT14 is significantly reduced when compared to HF-KC (FIG. 2D). Immunofluorescent staining further confirmed the expression of KRT19, KRT15, p63, KRT14, β-cat, LEF1, and Ki67 in hiPSC-EPSCs after 3 weeks in culture (p3), as shown in comparison to HF-KC (FIG. 2E). Significant nuclear accumulation of 3-Cat, a hallmark of Wnt signaling activation (Aoki et al., Proc Natl Acad Sci USA. 1999 Jan. 5, 96(1):139-44; Zhang et al., Int J Med Sci. 2013, 10(6):738-746) in hiPSC-EPSC but not in HF-KC (FIG. 2E, arrows), was found.

### Example 3: Activation of Wnt Signaling Pathway Via GSK3 Inhibition Enables Long-Term Expansion of hiPSC-EPSC

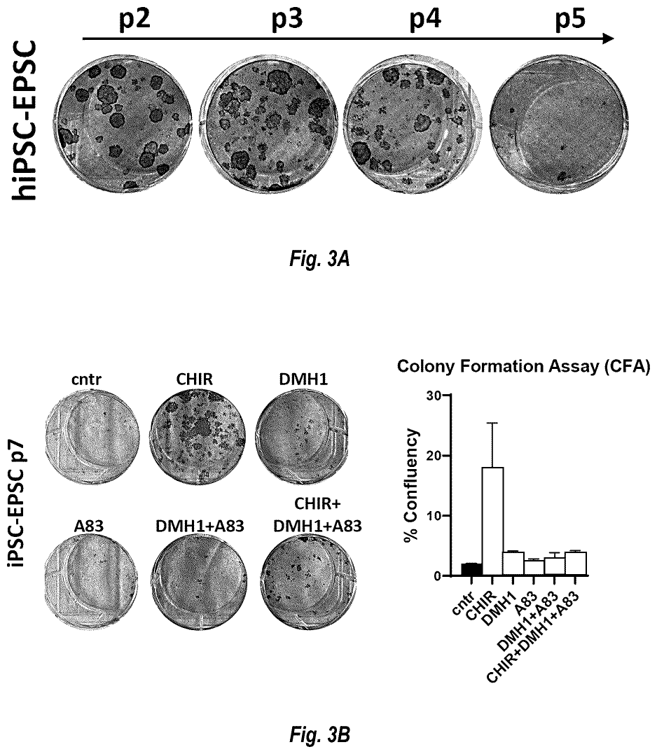

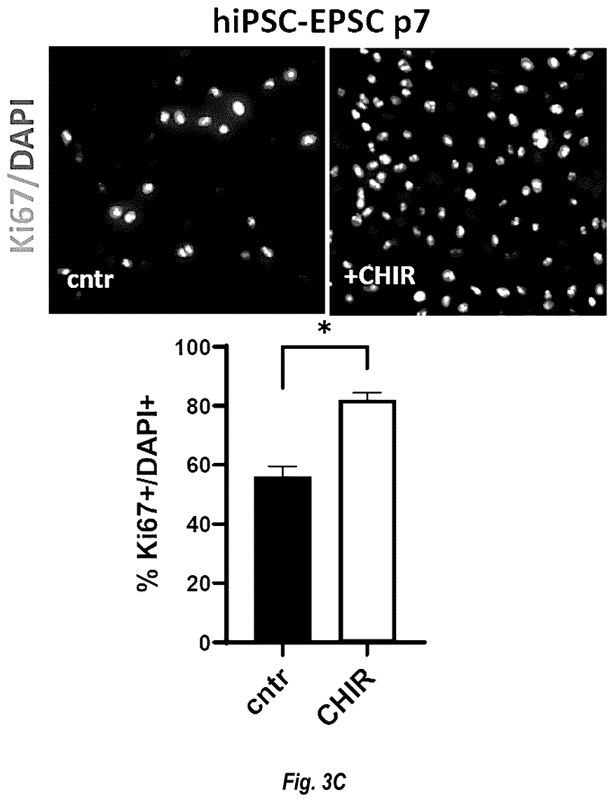

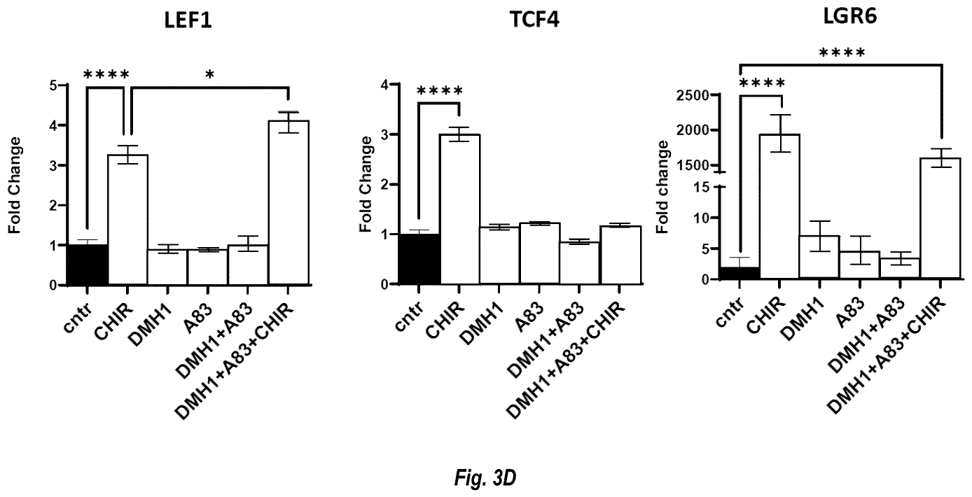

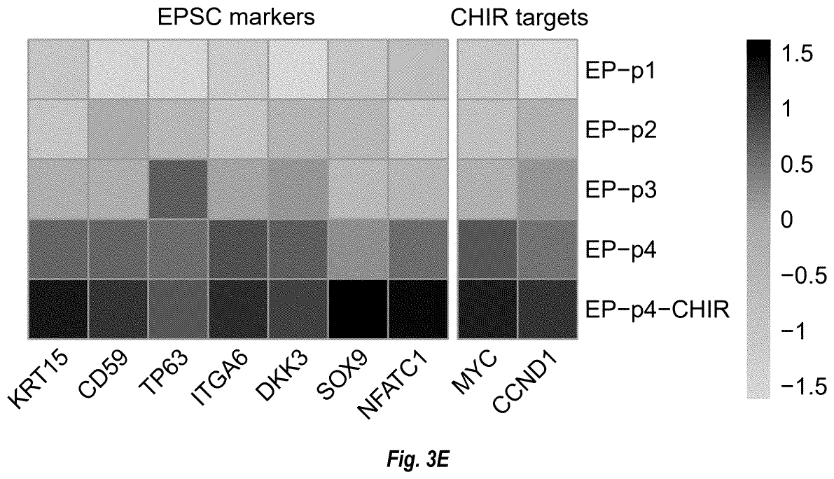

Despite EPSCs having substantial self-renewal capability in vivo, the prolonged expansion in vitro of EPSC isolated from various tissues has remained challenging. It is well documented that in conventional cultures, freshly isolated EPSCs rapidly lose their clonogenicity and stem cell marker expression signature. Inoue et al, Lab invest. 2009 August, 89(8):844-56; Ohyama et al., J Clin Invest. 2006 January, 116(1):249-60; Yang et al., Stem Cell Research & Therapy (2020) 11:303. As illustrated in FIG. 3A, the clonogenicity of hiPSC-EPSCs was diminishing as a result of serial passaging. In an effort to enable long-term propagation of iPSC-EPSCs, perturbagens were tested, including GSK-3 inhibitor and Wnt agonist (CHIR-99021), BMP inhibitor (DMH-1), and TGF-β inhibitor (A-83-01). The cells were cultured in the expansion medium either alone or with 2.5 μM A-83-01, 1 μM DMH-1, and 2.5 μM CHIR99021 separately or in various combinations. When small molecules were added to the cell culture media at passage 4 of the expansion process, the treatment with the GSK3 inhibitor and not BMP or TGF-β inhibitors, preserved clonogenicity of hiPSC-EPSCs (FIG. 3B). Immunofluorescent analysis using Ki67 antibodies revealed significantly higher number of proliferating cells withing hiPSC-EPSC populations treated with CHIR-99021 (FIG. 3C). Gene expression analysis by qPCR confirmed upregulation of Wnt pathway genes including LEF1, TCF4, and LGR6, consistent with the on-target effect of CHIR-99021 (FIG. 3D). Bulk RNA-seq was performed to evaluate the effect of CHIR-99021 on the HF bulge EPSC marker expression of passage 4 hiPSC-EPSCs. Importantly, CHIR-99021 treatment increased the expression of HF bulge EPSC markers including KRT15, CD59, TP63, ITGA6, DKK3, SOX9, and NFATC1 (FIG. 3E). These data suggest that CHIR-99021 not only supports long-term maintenance of hiPSC-EPSC proliferation, but also enhances EPSC features.

### Example 4: In Vitro Functional Analysis of hiPSC-EPSC in 3D Tissue Constructs

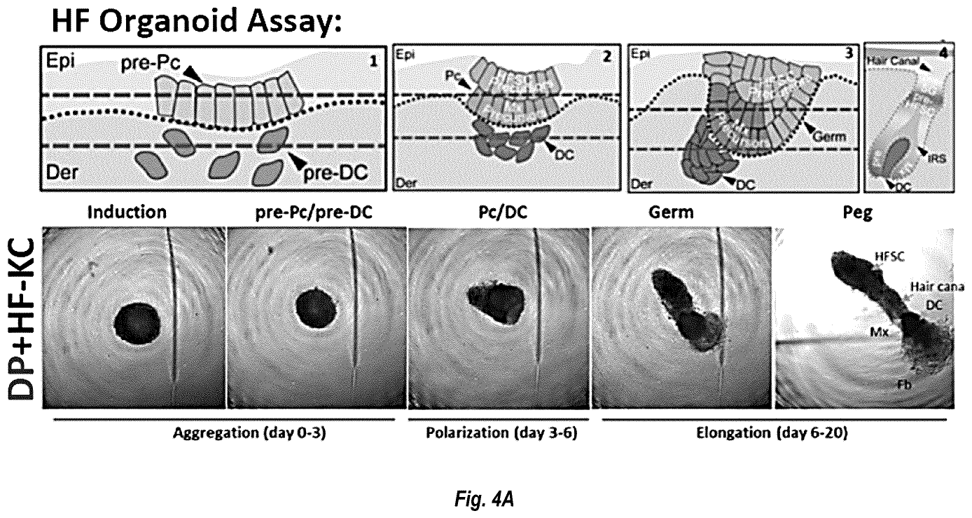

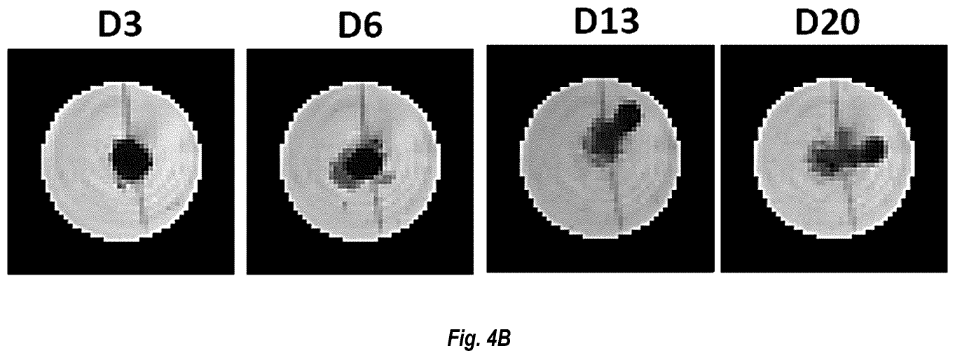

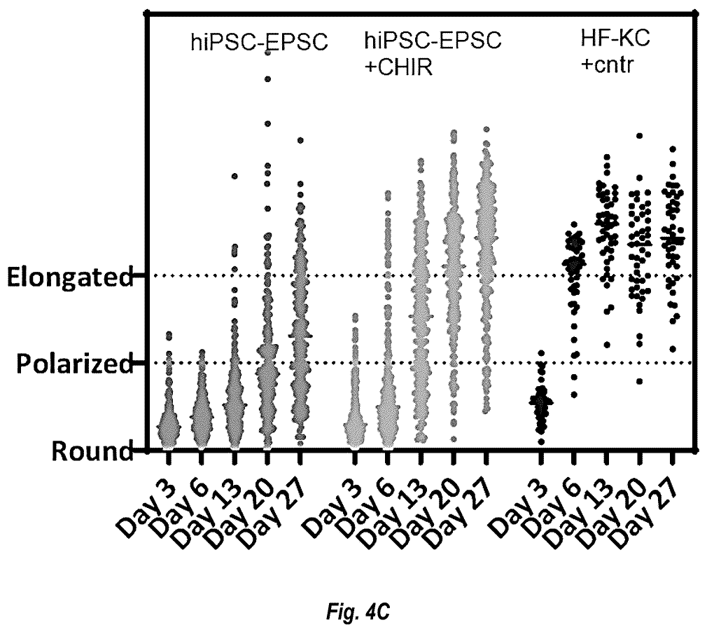

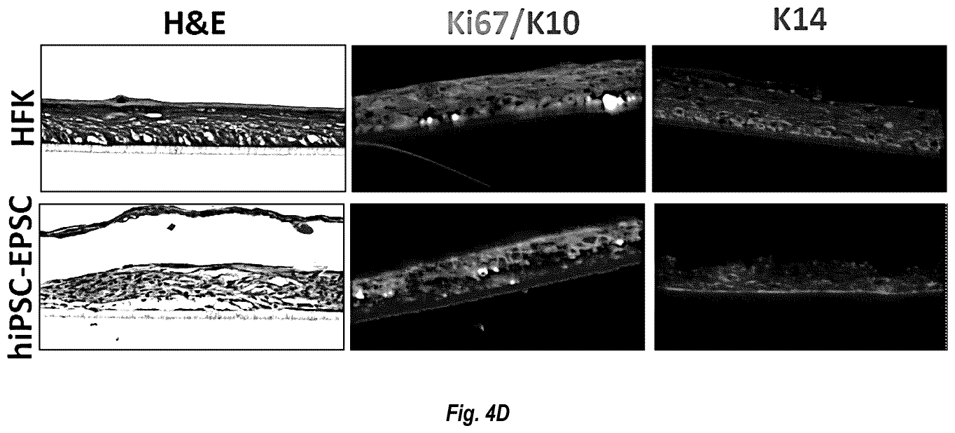

The stable hiPSC-EPSC with features of HF bulge EPSC described above were investigated as to whether they were receptive to specific mesenchymal stimuli and capable of creating complex multilayered tissues. The hiPSC-EPSC were incorporated into a 3D HF organoid assay. The 3D HF organoid assay is a high throughput screening assay that employs dermal papilla (DP) cells and HF-KC capable of self-assembling into elongated organoids resembling natural HF. See further details in the Materials & Methods section above. As illustrated in FIG. 4A, the self-organization process of HF organoids consists of three stages: aggregation, polarization and elongation, each approximating the major stages of folliculogenesis. When incorporated into HF organoid assay, iPSC-EPSCs were capable of recapitulating the key events of the HF morphogenesis by inducing an architecture similar to that of control HF-KC (FIG. 4B). To quantitively assess the organoids elongation and provide an indicator of hiPSC-EPSC functionality in vitro, an automated computational image-based analysis was employed that calculates the roundness of the generated 3D structures, assigning values between 1 and 0, where 1 represents a perfectly round object and 0 a long shaped one. By plotting time-lapse values of roundness score in a graph, the ability of iPSC-EPSC cells to generate elongated structure in combination with DP cells (FIG. 4C) was quantified. As expected, the percentage of elongated organoids was increased when GSK-3 inhibitor, CHIR-99021 (2.5 μM), was added to EPSC and organoid culture (FIG. 4C). To recapitulate the native skin tissue environment, 3D skin equivalents were generated using hiPSC-EPSCs in combination with foreskin derived fibroblasts (HFF) (FIG. 4B). HFFs were cultured in transwell for 14 days creating a thin dermal layer. Either hiPSC-EPSCs or foreskin derived keratinocytes (HFK) were then seeded onto the dermal layer, and tissues were grown at an air-liquid interface for an additional 7 days to compare their capacity to form stratified epithelium. See further details in the Materials & Methods section above. The tissues generated by hiPSC-EPSC demonstrated stratified epithelia with cellular architecture and marker expression similar to that generated by control HFKs (FIG. 4D). Immunofluorescent staining for epidermal differentiation markers showed that K10 (KRT10) was localized in the suprabasal layer, and K14 (KRT14) was localized in the basal and central layer of stratified epithelia. The expression of proliferation marker, Ki67, was restricted to the basal layer of the stratified tissue. The findings indicate that hiPSC-EPSC are responsive to inductive mesenchymal signals and capable of generating complex stratified tissues in vitro.

### Example 5: In Vivo Transplantation of hiPSC-EPSC Generates New Hair Follicles

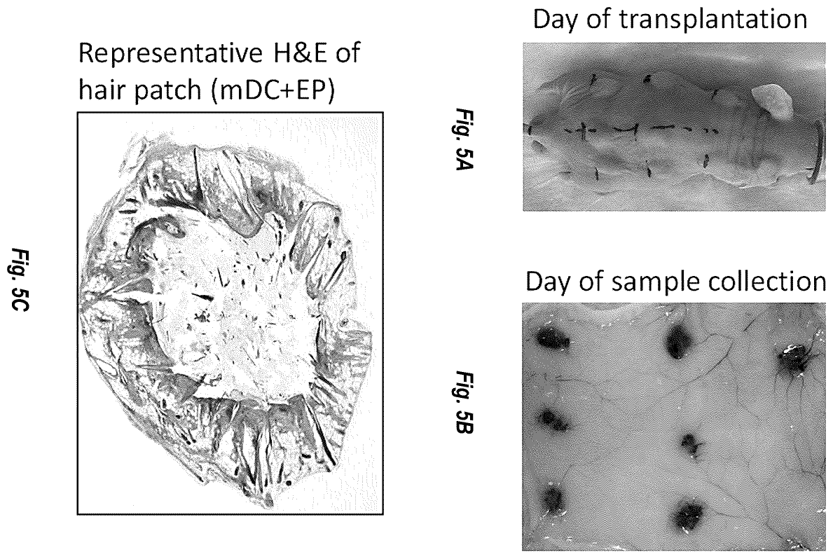

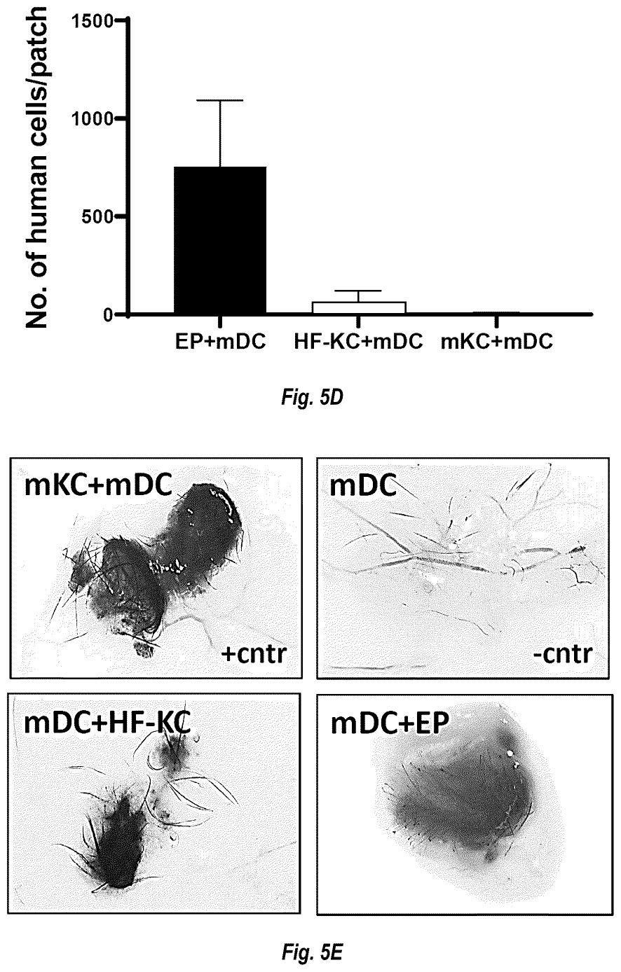

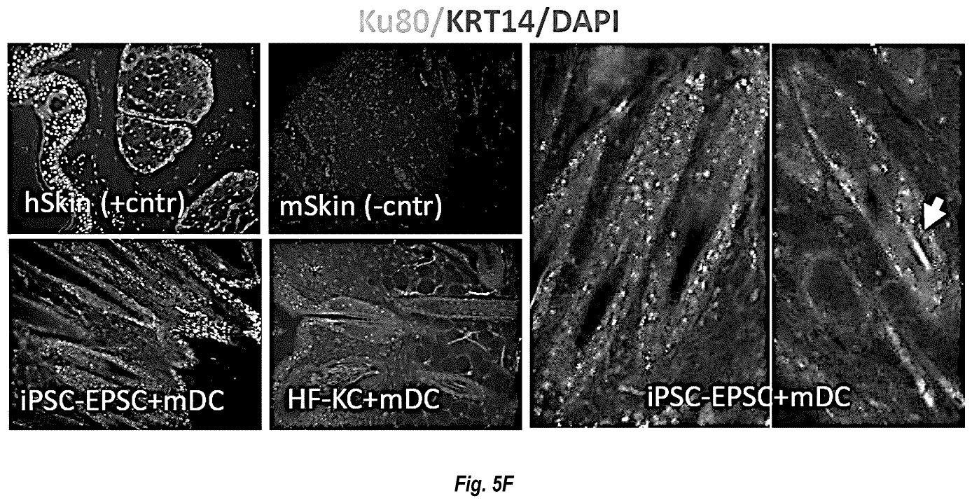

hiPSC-EPSC that accurately mimic the phenotype of native HF bulge EPSC should be able to reconstitute the epithelial components of the hair follicle and interfollicular epidermis. Therefore, mouse hair patch assays were employed to further confirm the regenerative capacity of hiPSC-EPSCs and characterize the emerging hair. See further details in the Materials & Methods section above. The hiPSC-EPSCs or control HF-KC were combined with neonatal mouse dermal cells (mDC) and injected subcutaneously into the back skin of immune-deficient nude mice (FIG. 5A). The skin of the mice was excised and examined under a dissecting microscope 3 weeks after transplantation. Patches containing HFs were observed underneath the skin (FIG. 5B). Histological analysis revealed that the injected epithelial cells aggregated to form a cyst in the host subcutis. Hair follicles growing outward from the cyst were evident (FIG. 5C).

Representative images of hair patches photographed under a stereomicroscope are shown in FIG. 5E. The hiPSC-EPSC combined with mDC were capable of forming large patches with multiple HFs while mDC injected alone formed much smaller patches containing only a few HFs. A larger number of HFs were apparent in the hair patches generated with hiPSC-EPSC when compared with the patches generated with HF-KC (FIG. 5E). The human origin and the number of the epithelial cells in hair patches was confirmed by a quantitative Alu-qPCR assay with a human-specific Alu-repeat sequence primer. The Alu-qPCR analysis revealed that HFs formed with hiPSC-EPCS retained higher number of human cells as illustrated in FIG. 5D.

Immunofluorescent analysis was conducted with different antibodies. See further details in the Materials & Methods section above. Immunofluorescent analysis with antibodies against human-specific nuclear protein Ku80 and human-specific KRT14 confirmed that the HF epithelium and the stratified epithelium of the cyst were of human origin and the surrounding mesenchymal components were composed of mouse cells (FIG. 5E). The HF-KC formed smaller patches containing fewer HFs and consistent with the Alu-qPCR data, the immunoreactivity of those cells was reduced when compared to hiPSC-EPSC. The in vivo findings confirm that EPSC from hiPSC are capable of generating epithelial component of HFs.

Taken together, these results confirm the receptivity to trichogenic dermal signals and potent hair inductive capacity of hiPSC-EPSC.

What is claimed is:

1. A method of producing epithelial stem cells (EPSCs) from human induced pluripotent stem cells (hiPSCs), wherein the method comprises:
(a) culturing hiPSCs in a medium comprising retinoic acid (RA);
(b) culturing the cells of step (a) in a medium comprising RA, bone morphogenetic protein 4 (BMP4), and epidermal growth factor (EGF);
(c) culturing the cells of step (b) in a medium comprising a keratinocyte serum-free medium, RA, BMP4, EGF, and ROCK inhibitor; and
(d) culturing the cells of step (c) in a medium comprising a keratinocyte serum-free medium, BMP4, EGF, and Rock inhibitor, thereby producing EPSCs.

2. The method of claim 1, wherein the culturing steps (a), (b), (c), and (d) are conducted for a duration independently selected from about 1, 2, 3, or more days.

3. The method of claim 2, wherein the culturing steps (a), (b), (c), and (d) are each conducted for 2 days.

4. A method of producing epithelial stem cells (EPSCs) from human induced pluripotent stem cells (hiPSCs), wherein the method comprises:
(a) culturing hiPSCs in a medium comprising retinoic acid (RA) for 2 days;
(b) culturing the cells of step (a) in a medium comprising RA, bone morphogenetic protein 4 (BMP4), and epidermal growth factor (EGF) for 2 days;
(c) culturing the cells of step (b) in a medium comprising RA, BMP4, EGF, and ROCK inhibitor for 2 days; and
(d) culturing the cells of step (c) in a medium comprising BMP4, EGF, and Rock inhibitor for 2 days.

5. The method of any one of the preceding claims, wherein the medium of culturing step (a) does not include BMP4.

6. The method of any one of the preceding claims, wherein the medium of culturing step (a) further comprises a keratinocyte serum-free medium and a matrix comprising a solubilized basement membrane matrix secreted by Engelbreth-Holm-Swarm (EHS) mouse sarcoma cells and human Collagen Type I (BMM/Coll1 matrix).

7. The method of any one of the preceding claims, wherein the medium of culturing step (b) further comprises the BMM/Coll1 matrix.

8. The method of any one of the preceding claims, wherein the medium of culturing step (c) further comprises human Collagen type I, fibronectin, and laminin-521 (Coll1/FN/LN521).

9. The method of any one of the preceding claims, wherein the medium of culturing step (d) further comprises Coll1/FN/LN521.

10. The method of any one of the preceding claims, wherein the medium used in culturing steps (a)-(d) is feeder-free and serum-free.

11. A method of producing epithelial stem cells (EPSCs) from human induced pluripotent stem cells (hiPSCs) comprising:
differentiating hiPSCs into EPSCs using a feeder-free and serum-free system comprising retinoic acid (RA), bone morphogenetic protein 4 (BMP4), epidermal growth factor (EGF), and ROCK inhibitor.

12. The method of any one of the preceding claims, wherein the EPSCs express tumor protein p63 (TP63) and keratin 15 (KRT15).

13. The method of any one of the preceding claims, further comprising culturing the hiPSCs for one or more days with:
i. a BMM/Coll1 matrix; and
ii. a serum-free medium comprising basal medium, recombinant human basic fibroblast growth factor (rh bFGF), and recombinant human transforming growth factor β (rh TGFβ).

14. The method of any one of the preceding claims, wherein the method further comprises expanding the EPSCs by maintaining the cells on a feeder-free medium.

15. The method of claim 14, wherein the feeder-free maintaining step comprises:
(a) culturing in a first feeder-free medium comprising BMP4, EGF, and ROCK inhibitor; and
(b) maintaining the cells of step (a) in a second feeder-free medium comprising EGF and ROCK inhibitor, optionally wherein the second feeder-free medium does not comprise BMP4.

16. The method of claim 15, wherein culturing step (a) and maintaining step (b) are each conducted for 1, 2, 3, or more days, which may be the same or different number of days.

17. The method of claim 16, wherein culturing step (a) and maintaining step (b) are both conducted for 2 days.

18. The method of any one of claims 15 to 17, wherein the feeder-free medium of culturing step (a) further comprises Coll1/FN/LN521 and a keratinocyte serum-free medium.

19. The method of any one of claims 15 to 18, wherein the feeder-free medium of maintaining step (b) further comprises a keratinocyte serum-free medium.

20. The method of any one of claims 14 to 19, wherein the expanding step is for at least 2, least 3, at least 4, at least 5, or at least 6 passages.

21. The method of the immediately preceding claim, wherein the expanding step is for 6 passages.

22. The method of any one of claims 1-13, wherein the method further comprises expanding the EPSCs by maintaining the cells on a feeder medium comprising mitotically inactivated 3T3-J2 mouse fibroblasts.

23. The method of the immediately preceding claim, wherein feeder medium further comprises ROCK inhibitor.

24. The method of claim 22 or claim 23, wherein the feeder medium further comprises Coll1/FN/LN521 and a keratinocyte growth medium.

25. The method of the immediately preceding claim, wherein the keratinocyte growth medium comprises DMEM/F12, dipeptide L-alanine-L-glutamine, keratinocyte growth medium 2 (KG2) basal medium, fetal bovine serum (FBS), adenine, an effective amount of one or more antibiotics and/or antimycotics, hydrocortisone, cholera toxin (CT), epidermal growth factor (EGF), and insulin.

26. The method of any one of claims 22 to 25, wherein the feeder medium further comprises DMEM, bovine calf serum (BCS), one or more antibiotics and/or antimycotics, and L-Glutamine.

27. The method of claim 25 or claim 26, wherein the one or more antibiotics and/or antimycotics comprise penicillin, streptomycin, and amphotericin B.

28. The method of any one of the preceding claims, wherein the EPSCs have increased expression of markers TP63 and KRT15 after/during expansion as compared to iPSC controls.

29. The method of any one of claims 6-10 and 13-28, wherein BMM/Coll1 matrix is formed by:
(a) combining BMM with DMEM/F12 to form a diluted BMM solution;
(b) adding human Collagen Type I (Coll1) solution to the diluted BMM solution to form a BMM/Coll1 matrix.

30. The method of any one of the proceeding claims, wherein the method further comprises culturing the EPSCs with a perturbagen.

31. The method of claim 30, wherein the perturbagen is a GSK-3 inhibitor and/or Wnt agonist.

32. The method of claim 31, wherein the GSK-3 inhibitor and/or Wnt agonist is CHIR-99021.

33. The method of any one of the preceding claims, wherein the EPSC expresses at least two, three, four, or each of CD59, CD99, LEF1, CTNNB1, FZD1, LGR5, DCT, TCF4, and LGR6.

34. The method of any one of the preceding claims, wherein the EPSC expresses at least two, three, four, five, six, seven, eight, nine, or each of CD59, CD99, KRT15, KRT19, DKK3, FZD1, FZD2, CTNNB1, TGFB2, FST, DCN, SOX9, NFATC1, ITGA6, ITGB1, LEF1, TCF4, TP63, and LGR6.

35. An epithelial stem cell (EPSC) produced by the method of any one of the preceding claims.

36. The EPSC of the immediately preceding claim, wherein the EPSC expresses one or more of CD59, CD200, KRT15, KRT19, DKK3, FZD1, FZD2, CTNNB1, TCF4, LEF1, LGR6, TGFB2, SOX9, NFATC1, and DCN at an increased level relative to a naturally occurring EPSC, and/or one or more of ITGA6, ITGB1, FST, KRT14, and TP63 at a decreased level relative to a naturally occurring EPSC.

37. An EPSC differentiated from an induced pluripotent stem cell, wherein the EPSC expresses one or more of CD59, CD200, KRT15, KRT19, DKK3, FZD1, FZD2, CTNNB1, TCF4, LEF1, LGR6, TGFB2, and DCN at an increased level relative to a naturally occurring EPSC, and/or one or more of ITGA6, ITGB1, FST, KRT14, and TP63 at a decreased level relative to a naturally occurring EPSC.

38. The EPSC of claim 36 or 37, wherein the EPSC expresses two, three, four, five, six, seven, eight, nine, ten, or each of CD59, CD200, KRT15, KRT19, DKK3, FZD1, FZD2, CTNNB1, TCF4, LEF1, LGR6, TGFB2, and DCN at an increased level relative to a naturally occurring EPSC.

39. The EPSC of any one of claims 36-38, wherein the EPSC expresses two, three, or each of ITGA6, ITGB1, FST, KRT14, and TP63 at a decreased level relative to a naturally occurring EPSC.

40. The EPSC of any one of claims 36-39, wherein the naturally occurring EPSC is a bulge hair follicle-derived ketatinocyte.

41. The EPSC of any one of claims 36-40, which is a human EPSC.

42. A method of culturing the EPSC of any one of claims 35-41 under conditions to obtain hair follicles and/or interfollicular epidermis.

43. An organoid produced using the EPSCs of any one of claims 35-41.

44. The organoid of the immediately preceding claim, wherein the organoid is a folliculogenic organoid.

45. A method of producing hair follicles comprising culturing the EPSCs of any one of claims 35-41 with fibroblasts and/or dermal papilla cells to obtain hair follicles.

46. A method of producing a skin graft comprising culturing the EPSCs of any one of claims 35-41 under conditions to produce a skin graft.

47. A method of producing a skin graft comprising culturing the EPSCs of any one of claims 35-41 under conditions to produce a cultured epidermal graft.

48. Use of the EPSCs of any one of claims 35-41 to obtain hair follicles and/or interfollicular epidermis.

49. Use of the EPSCs of any one of claims 35-41 for a replacement skin graft.

50. The method of claim 47 or the use of claim 49, comprising delivering the EPSCs onto a wound bed, optionally wherein delivering comprises spraying.

51. The method or use of claim 49 or 50, wherein the EPSCs are expanded in culture, optionally wherein the expansion occurs before delivering the EPSCs onto a wound bed.

52. The method or use of claim 51, wherein the EPSCs are cultured with a dermal component, optionally wherein the dermal component comprises dermal fibroblasts and/or a collagen substrate.

53. A method of growing a hair follicle, comprising:
(a) loading one or more EPSCs according to any one of claims 35-41 into a cellular scaffold; and
(b) implanting the cellular scaffold into dermal tissue.

54. The method of claim 53, wherein the cellular scaffold is in the form of a hydrogel, a fiber network, or a photoinitiated 3D structure.

55. The method of claim 53 or claim 54, wherein the cellular scaffold comprises a biodegradable polymer.

56. The method of any one of claims 53-55, wherein the cellular scaffold comprises:
(a) a cell reservoir; and
(b) a guide attached to the cell reservoir comprising one or more first biodegradable polymers.

57. A method of growing a hair follicle, comprising implanting the organoid of claim 43 or claim 44 into dermal tissue.

58. The method of the immediately preceding claim, further comprising piercing the dermal tissue with a needle prior to implanting the organoid.

59. The method of claim 57 or claim 58, wherein a cellular scaffold containing the organoid is implanted into the dermal tissue.
